(19) United States
(12) Patent Application Publication
(10) Pub. No.: US 2023/0014008 A1
(43) Pub. Date: Jan. 19, 2023

(54) METHODS FOR IMPROVING SPATIAL PERFORMANCE

(71) Applicant: 10x Genomics, Inc., Pleasanton, CA (US)

(72) Inventor: Shankar Shastry, Pleasanton, CA (US)

(21) Appl. No.: 17/863,842

(22) Filed: Jul. 13, 2022

Related U.S. Application Data
(60) Provisional application No. 63/221,223, filed on Jul. 13, 2021.

(51) Int. Cl. C12Q 1/6837; C12Q 1/682

(52) U.S. Cl. CPC C12Q 1/6837; C12Q 1/682

(57) ABSTRACT

Disclosed herein are compositions and methods for determining a presence or abundance of an analyte in a biological sample. The methods disclosed herein include: (a) providing a biological sample on a substrate comprising a plurality of capture probes, wherein a capture probe of the plurality of capture probes comprises a capture domain; (b) releasing the analyte from the biological sample; (c) affixing a stretching moiety to the analyte; (d) hybridizing the analyte to the capture domain of the capture probe; (e) applying a stretching force to the stretching moiety, thereby elongating the analyte hybridized to the capture domain; and (f) generating an extended capture probe using the analyte as a template.

### CROSS-REFERENCE TO RELATED APPLICATIONS

This application claims the benefit of U.S. Provisional Application Ser. No. 63/221,223, filed Jul. 13, 2021, the entire contents of which are incorporated by reference herein.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or that provide significant analyte data from individual, single cells, but fails to provide information regarding the position of the single cells from the originating biological sample (e.g., tissue).

Target nucleic acid analytes released from the cells of a biological sample can include secondary structure which prevent binding and reverse transcription of the target nucleic acid analyte and extension of a capture domain therefrom, which can lead to a decrease in resolution or performance of spatial analysis of target nucleic acid analytes in a biological sample. Methods to resolve secondary structure present in target nucleic acid analytes are needed.

### SUMMARY

Resolving the secondary structure of target analytes for spatial analysis methods would be beneficial in a number of ways. For example, the probability that a polynucleotide target analyte includes secondary structure increases with length and number of complementary regions present in the target analyte sequence. Potential secondary structures include coiling, stem-loops, pseudo-knots, or alternative helix structures (e.g., A-, of Z-form helix structures).

The secondary structures present in a target analyte can be resolved through the application of a stretching force on the target analyte. A stretching force is a force applied to the target analyte capable of resolving low energy bonds in the target analyte, such as helix coiling or base pairs involved in stem-loops. The stretching force is applied to one end of the target analyte while the other end is bound to a capture domain of a capture probe, which is further affixed to a substrate for determining the spatial location of the target analytes. Applying the stretching force to the target analyte extends the nucleic acid backbone and the force is applied to the secondary structures. Applying sufficient stretching force resolves the target analyte's secondary structure that would otherwise prevent or limit the target analyte's use as a template (e.g., in reverse transcription, elongation, amplification).

The capture domain of the capture probe is extended through transcription according to the sequence of the linearized target analyte, thereby creating an extended capture probe (also termed extended ligation product where relevant). The extended capture probe, or a complement thereof, can then be released from the substrate and the target analyte sequence amplified and spatial location determined according to known spatial transcription methods.

In particular, methods for the analysis of nucleic acid analytes in a biological sample are described herein. For example, provided herein is a method for determining a presence or abundance of a nucleic acid analyte in a biological sample, the method comprising: (a) providing the biological sample on a substrate comprising a plurality of capture probes, wherein a capture probe of the plurality of capture probes includes a spatial barcode and a capture domain; (b) releasing the nucleic acid analyte from the biological sample; (c) affixing a stretching moiety to the nucleic acid analyte; (d) hybridizing the nucleic acid analyte to the capture domain; (e) applying a stretching force to the stretching moiety, thereby elongating the nucleic acid analyte hybridized to the capture domain; and (f) generating an extended capture probe using the nucleic acid analyte as a template.

In some embodiments, the method can further include determining (i) all or a part of a sequence of the nucleic acid analyte or a complement thereof, and (ii) the spatial barcode or a complement thereof, and using the determined sequences of (i) and (ii) to determine the presence or abundance of the nucleic acid analyte in the biological sample. The determining step can include sequencing (i) all or a part of a sequence of the nucleic acid analyte or a complement thereof, and (ii) spatial barcode or a complement thereof. The sequencing can be high throughput sequencing.

In a second aspect, the disclosure includes a method for determining a presence or abundance of a nucleic acid analyte in a biological sample, the method comprising:(a) providing the biological sample on a substrate comprising a plurality of capture probes, wherein a capture probe of the plurality of capture probes includes a spatial barcode and a capture domain; (b) releasing the nucleic acid analyte from the biological sample; (c) affixing a stretching moiety to the nucleic acid analyte; (d) hybridizing the nucleic acid analyte to the capture domain; (e) applying a stretching force to the stretching moiety, thereby elongating the nucleic acid analyte hybridized to the capture domain; (f) hybridizing a padlock oligonucleotide to the analyte hybridized to the capture domain, wherein the padlock oligonucleotide includes: (i) a first sequence that is substantially complementary to a first portion of the nucleic acid analyte, or a complement thereof, (ii) a backbone sequence, and (iii) a second sequence that is substantially complementary to a second portion of the nucleic acid analyte, or a complement thereof; (g) ligating the first sequence to the second sequence of the padlock oligonucleotide, thereby generating a circularized padlock oligonucleotide; (h) amplifying the circularized padlock oligonucleotide, thereby creating an amplified circularized padlock oligonucleotide, and (i) identifying the presence or abundance of the nucleic acid analyte in the biological sample.

In some embodiments, the identifying the presence or abundance of the nucleic acid analyte can include determining (i) all or a part of a sequence of the nucleic acid analyte or a complement thereof, and (ii) the spatial barcode or a complement thereof, and using the determined sequences of (i) and (ii) to determine the presence or abundance of the nucleic acid analyte in the biological sample.

The identifying the presence or abundance of the nucleic acid analyte can include detecting a signal corresponding to the amplified circularized padlock oligonucleotide on the substrate. The amplifying the circularized padlock oligonucleotide can include rolling circle amplification. In some embodiments, the method can further include quantitating the signal.

In some embodiments, the first sequence of the padlock oligonucleotide and the second sequence of the padlock oligonucleotide can be substantially complementary to adjacent sequences of the nucleic acid analyte. The first sequence of the padlock oligonucleotide and the second sequence of the padlock oligonucleotide can be substantially complementary to sequences of the nucleic acid analyte that are not adjacent to one another, generating a gap between the first sequence and the second sequence upon hybridization of the first sequence and the second sequence to the nucleic acid analyte, wherein the gap can be filled using a polymerase. The ligating step can include enzymatic ligation or chemical ligation. The enzymatic ligation can utilize T4 DNA ligase.

In some embodiments, the stretching moiety can be a magnetic bead, and the stretching force can be a magnetic force.

The stretching force can be a linear force orthogonal to a plane of an upper surface of the substrate, a rotational force around a rotational axis orthogonal to the plane of the upper surface of the substrate, or both. In some embodiments, the affixing the stretching moiety to the nucleic acid analyte can include affixing a first binding moiety to a second binding moiety, where the stretching moiety can include the first binding moiety, and where the nucleic acid analyte can include the second binding moiety associated with a 5′ end of the nucleic acid analyte or a 3′ end of the nucleic acid analyte. The first binding moiety can include digoxigenin, anti-digoxigenin, biotin, avidin, or streptavidin. The second binding moiety can include digoxigenin, anti-digoxigenin, biotin, avidin, or streptavidin.

The stretching moiety further can include a cleavable linker.

The stretching force can be in a range from 0.05 piconewtons (pN) to 100 pN. The range can be from 0.1 pN to 0.5 pN. The range can be from 0.2 pN to 0.4 pN. The stretching force can be applied for about 1 second (s) to about 10 minutes (min), from about 30 s to about 5 min, or from about 1 min to about 3 min. The stretching force can be applied using one of a magnetic field, an electric field, or a light field. The stretching force can be a modulated stretching force. The method can further include releasing the extended capture probe from the substrate. The releasing the nucleic acid analyte from the biological sample can include treating the biological sample with a solution comprising pepsin or proteinase K.

In some embodiments, the capture domain can include a poly(T) sequence. The nucleic acid analyte can be an RNA. The RNA can be mRNA. The nucleic acid analyte can be DNA. The DNA can be genomic DNA. The biological sample can be a tissue sample. The tissue sample can be a fixed tissue sample. The fixed tissue sample can be a formalin-fixed paraffin-embedded (FFPE) sample. The tissue sample can be a fresh tissue sample or a frozen tissue sample.

In a third aspect, the disclosure includes a kit, comprising:(a) a plurality of stretching moieties; (b) a plurality of primers; (c) one or more enzymes selected from a polymerase, a reverse transcriptase, and a ligase; (d) a substrate comprising a plurality of capture probes, wherein a capture probe of the plurality of capture probes includes a spatial barcode and a capture domain; and (e) instructions for performing a method disclosed herein.

In another aspect, the disclosure provides a method for determining a presence or abundance of a nucleic acid analyte in a biological sample, the method comprising: (a) providing the biological sample on a first substrate; (b) aligning the first substrate with a second substrate comprising an array, such that at least a portion of the biological sample is aligned with at least a portion of the array, where the array comprises a plurality of capture probes, where a capture probe of the plurality of capture probes comprises a spatial barcode and a capture domain; (c) releasing the nucleic acid analyte from the biological sample, such that the nucleic acid analyte actively or passively migrates toward the capture probe, and binds the capture probe; (d) affixing a stretching moiety to the nucleic acid analyte; (e) hybridizing the nucleic acid analyte to the capture domain; (f) applying a stretching force to the stretching moiety, thereby elongating the nucleic acid analyte hybridized to the capture domain; and (g) extending the capture probe using the nucleic acid analyte as a template, thereby generating an extended capture probe.

The method can further comprise determining (i) all or a part of a sequence of the nucleic acid analyte or a complement thereof, and (ii) the spatial barcode or a complement thereof, and using the determined sequences of (i) and (ii) to determine the presence or abundance of the nucleic acid analyte in the biological sample. In some embodiments, the nucleic acid analyte is RNA or DNA.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “binding pair” refers to a pair of moieties which form bonded pairs when contacted. This can include high-affinity protein-ligand interactions, nucleotide base pairing, and antigen-antibody pairing. These pairs can be influenced by non-covalent intermolecular interactions such as hydrogen bonding, electrostatic interactions, hydrophobic and Van der Waals forces between the pair.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the cell.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A, 6B, and 6C are schematics illustrating how streptavidin cell tags can be utilized in an array-based system to produce spatially-barcoded cells or cellular contents.

FIGS. 7A and 7B are exemplary schematic diagrams showing attachment of a stretching moiety to an analyte.

FIG. 7C is a schematic diagram showing binding the analyte to a capture probe affixed to a substrate.

FIGS. 7D and 7E are schematic diagrams showing applying a stretching force to the stretching moiety to resolve the secondary structure of the analyte.

FIG. 7F is a schematic diagram showing extending the capture domain of the capture probe according to the sequence of the analyte.

FIG. 8A is a schematic diagram showing a padlock oligonucleotide.

FIG. 8B is a schematic diagram of an exemplary padlock oligonucleotide hybridized to a captured analyte bound to a substrate.

FIG. 8C is a schematic diagram of an exemplary amplification primer hybridized to an exemplary circularized padlock oligonucleotide.

FIG. 9 shows an exemplary spatial analysis workflow in which stretching moieties and a stretching force are applied to an analyte.

FIG. 10 shows an exemplary spatial analysis workflow in which stretching moieties and a stretching force are applied to an analyte and the presence of the analyte detected using padlock oligonucleotides.

FIG. 11 is a schematic diagram depicting an exemplary sandwiching process between a first substrate comprising a biological sample and a second substrate comprising a spatially barcoded array.

FIG. 12A provides perspective view of an exemplary sample handling apparatus 1400 in a closed position.

FIG. 12B provides a perspective view of the exemplary sample handling apparatus 1400 in an open position.

FIG. 13A shows an exemplary sandwiching process where a first substrate and a second substrate are brought into proximity with one another.

FIG. 13B shows a fully formed sandwich configuration creating a chamber formed from one or more spacers, a first substrate, and a second substrate.

FIGS. 14A-14C depict a side view and a top view of an exemplary angled closure workflow for sandwiching a first substrate and a second substrate. FIG. 14A depicts the first substrate angled over (superior to) the second substrate. FIG. 14B shows that as the first substrate lowers, and/or as the second substrate rises, the dropped side of the first substrate may contact the drop of the reagent medium. FIG. 14C depicts a full closure of the sandwich between the first substrate and the second substrate with the spacer contacting both the first substrate and the second substrate.

FIGS. 15A-15E depict an exemplary workflow for an angled sandwich assembly. FIG. 15A shows a substrate 1712 positioned and placed on a base with a side of the substrate supported by a spring. FIG. 15B depicts a drop of reagent medium placed on the substrate. FIG. 15C shows another substrate 1706 positioned above (superior to) substrate 1712 and at an angle substantially parallel with the base. In FIG. 15D, substrate 1706 is lowered toward the substrate 1712 such that a dropped side of the substrate 1706 contacts the drop first. FIG. 15E depicts a full sandwich closure of the substrate 1706 and the substrate 1712 with the drop of reagent medium positioned between the two sides.

FIG. 16A is a side view of an angled closure workflow.

FIG. 16B is a top view of an angled closure workflow.

### DETAILED DESCRIPTION

I. Introduction

Provided herein are methods for determining the presence and/or abundance of an analyte (e.g., a target analyte) in a biological sample, wherein the target analyte includes secondary structures. As used herein, the terms “analyte” and “target analyte” and like terms are interchangeable unless noted. In some embodiments, secondary structures within target analytes prevent binding, and/or transcription of at least a portion of the target analyte sequence on a spatial array during spatial array methods. Attaching a portion of the target analyte to a capture probe affixed to a substrate and applying a stretching force to the target analyte elongates the target analyte and eliminates present secondary structure. The stretching force is applied through a stretching moiety attached to one end of the target analyte responsive to an applied field. A field application instrument creates an applied field which creates the stretching force having a magnitude and direction. The stretching force reduces or eliminates the secondary structure of the target analyte and facilitates extension of the capture probe to provide an extended capture probe that includes a copy or complement of the stretched target analyte.

In some embodiments, the target analyte is contacted with a padlock oligonucleotide after the secondary structure is eliminated. Contacting the target analyte with the padlock oligonucleotide circularizes the padlock oligonucleotide. The circularized padlock oligonucleotide is contacted with an amplification primer and a portion of the sequence of the circularized padlock oligonucleotide is amplified. The amplified circularized padlock oligonucleotide sequence is contacted with a plurality of detection moieties thereby facilitating the detection and quantification of the target analyte on a spatial array that can be correlated back to the location of the analyte in a biological sample. Some embodiments of the methods result in higher levels of detected target analyte from a biological sample based on sequencing of the target analyte, or a complement thereof, released from the biological sample as compared to the levels of detected target analyte in which the secondary structure was not eliminated.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode, a ligation product) associated with the intermediate agent. Detection of the intermediate agent is therefore indicative of the analyte in the biological sample (e.g., cell, tissue section, etc.).

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198; U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621; WO 2018/091676, WO 2020/176788, WO 2022/061152, WO 2021/252747, WO 2022/140028; Rodrigues et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev D, dated October 2020), and/or the Visium Spatial Tissue Optimization Reagent Kits User Guide (e.g., Rev D, dated October 2020), both of which are available at the 10× Genomics Support Documentation website, and can be used herein in any combination. Further non-limiting aspects of spatial analysis methodologies and compositions are described herein.

Some general terminologies that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Examples of nucleic acid analytes include, but are not limited to, DNA (e.g., genomic DNA, cDNA) and RNA, including coding and non-coding RNA (e.g., mRNA, rRNA, tRNA, ncRNA).

Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

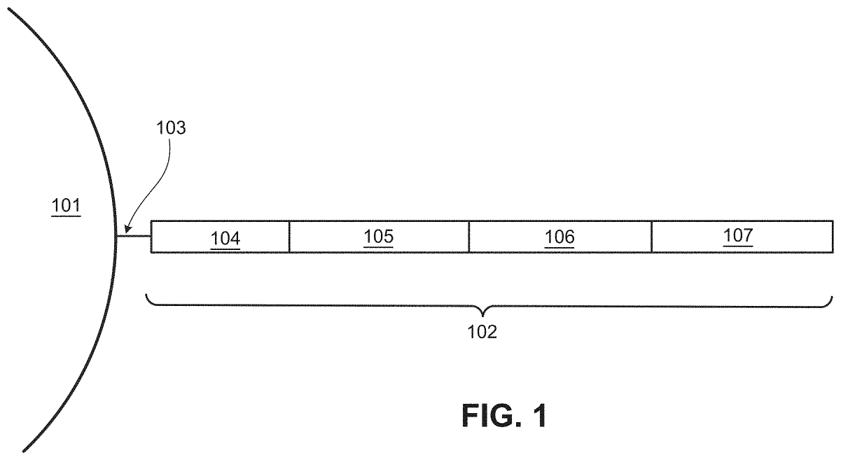

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

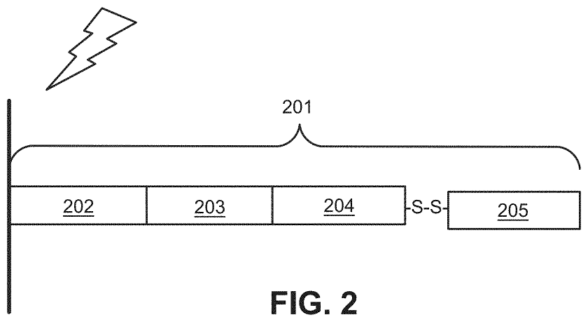

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

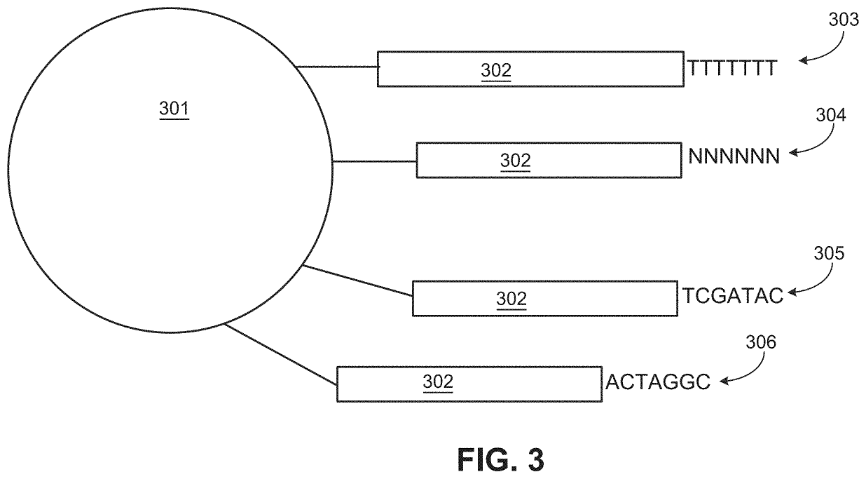

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

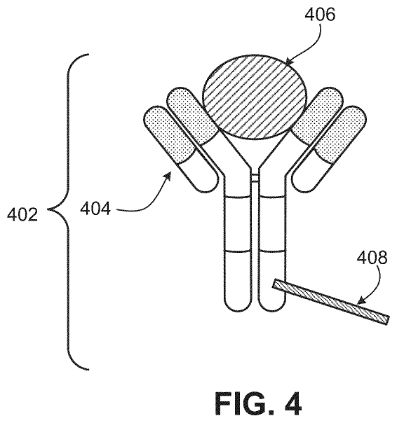

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

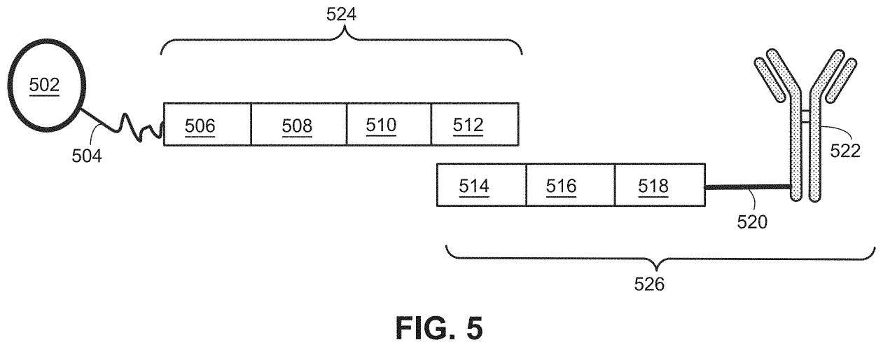

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

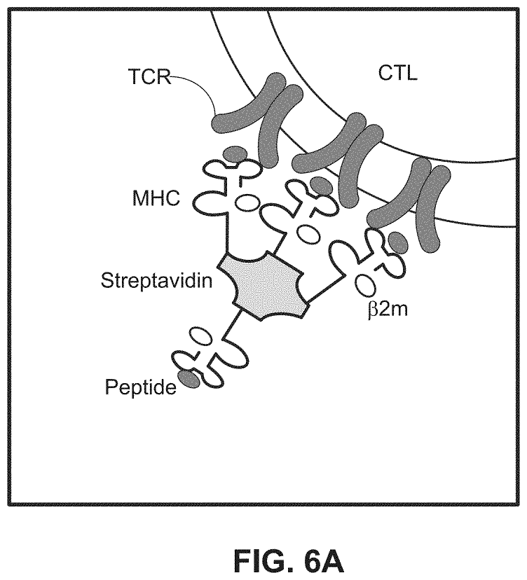

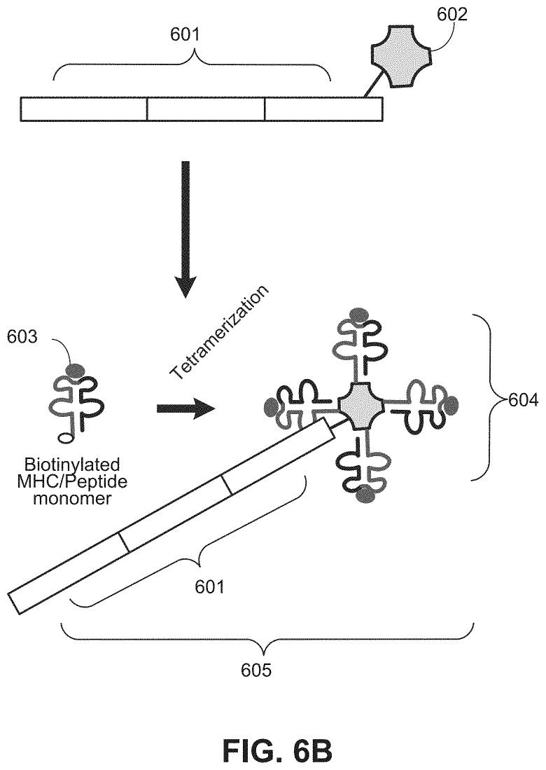

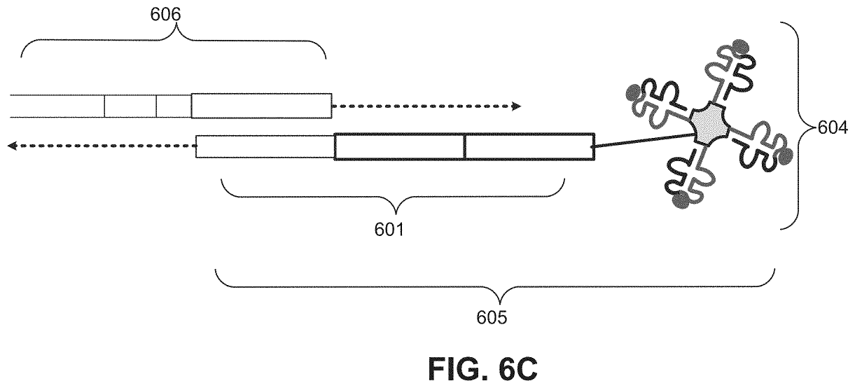

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MHC/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 605. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of the corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In general, spatial transcriptomics methods comprise a spatially-barcoded array populated with capture probes (as described further herein) that is contacted with a biological sample, the biological sample is permeabilized thereby allowing the analytes in the biological sample to migrate away from the sample and toward the array, for example via passive (e.g., gravitational) or active (e.g., electrophoretic) forces. The analyte hybridizes with a capture domain on a capture probe on the spatially-barcoded array. Once the analyte hybridizes/is bound to the capture domain of the capture probe, the capture probe is extended, using the capture analyte as a template, and the sequence of the extended capture probe, or a complement thereof, is analyzed to obtain spatially-resolved analyte information. The biological sample can also be optionally removed from the array following analyte capture on the array. In some instances, the spatially-barcoded array populated with capture probes (as described further herein) is contacted with a biological sample, and the biological sample is permeabilized, allowing the analyte to migrate away from the sample and toward the array. The analyte interacts with a capture probe on the spatially-barcoded array.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, generating an extended capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction). Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a nucleic acid molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663. Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev D, dated October 2020), and/or the Visium Spatial Tissue Optimization Reagent Kits User Guide (e.g., Rev D, dated October 2020). In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application Publ. No. WO 2021/102003 (from Appl. No. 2020/061064) and/or U.S. Patent Application Publ. No. US 2021-0150707 A1 (from Ser. No. 16/951,854), each of which is incorporated by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application Publ. No. WO 2021/067514 (filed as Appl. No. 2020/053655) and spatial analysis methods are generally described in WO 2020/061108 and/or U.S. Patent Application Publ. No. US 2021-0150707 A1 (from Ser. No. 16/951,864).

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application Publ. No. WO 2021/102005 (filed as PCT Appl. No. 2020/061066), and/or U.S. Patent Application Publ. No. US 2021-0158522 A1 (Filed as U.S. Ser. No. 16/951,843). Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Disclosed herein are methods for enhancing spatial detection of target analytes by resolving secondary structure of the analytes with stretching moieties. In some embodiments of the methods for resolving secondary structure of target analytes, one or more analytes from the biological sample are released from the biological sample and migrate to a substrate comprising an array of capture probes for attachment to the capture probes of the array. In some embodiments, the release and migration of the analytes to the substrate comprising the array of capture probes occurs in a manner that preserves the original spatial context of the analytes in the biological sample. In some embodiments, the biological sample is mounted on a first substrate and the substrate comprising the array of capture probes is a second substrate. In some embodiments, the method is facilitated by a sandwiching process.

Sandwiching methods have been described in WO 2022/140028, which is incorporated by reference in its entirety. Additional sandwiching processes are described in, e.g., US. Patent Application Pub. No. 20210189475, WO 2021/252747, and WO 2022/061152, each of which is incorporated by reference in its entirety. In some embodiments, the sandwiching process may be facilitated by a device, sample holder, sample handling apparatus, or system described in, e.g., US. Patent Application Pub. No. WO 2021/252747, PCT/US2021/036788, or PCT Publ. No. WO 2022/061152, each of which is incorporated by reference in its entirety.

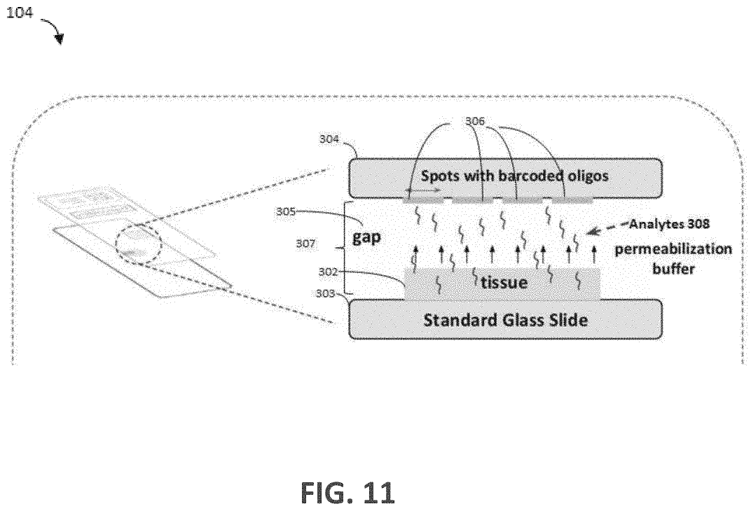

FIG. 11 is a schematic diagram depicting an exemplary sandwiching process 104 between a first substrate comprising a biological sample (e.g., a tissue section 302 on a slide 303) and a second substrate comprising a spatially barcoded array, e.g., a slide 304 that is populated with spatially-barcoded capture probes 306. During the exemplary sandwiching process, the first substrate is aligned with the second substrate, such that at least a portion of the biological sample is aligned with at least a portion of the array (e.g., aligned in a sandwich configuration). As shown, the second substrate (e.g., slide 304) is in a superior position to the first substrate (e.g., slide 303). In some embodiments, the first substrate (e.g., slide 303) may be positioned superior to the second substrate (e.g., slide 304). A reagent medium 305 (e.g., permeabilization solution) within a gap 307 between the first substrate (e.g., slide 303) and the second substrate (e.g., slide 304) creates a permeabilization buffer which permeabilizes or digests the sample 302 and the analytes (e.g., protein and/or nucleic acid (e.g., DNA or RNA), such as mRNA transcripts) 308 of the biological sample 302 may release, actively or passively migrate (e.g., diffuse) across the gap 307 toward the capture probes 306, and bind on the capture probes 306.

After the analytes (e.g., protein and/or nucleic acid (e.g., DNA or RNA)) 308 bind the capture probes 306, an extension reaction may occur, thereby generating a spatially barcoded library. For example, in the case of mRNA transcripts, reverse transcription may be used to generate a cDNA library associated with a particular spatial barcode. Barcoded cDNA libraries may be mapped back to a specific spot on a capture area of the capture probes 306. This data may be subsequently layered over a high-resolution microscope image of the biological sample, making it possible to visualize the data within the morphology of the tissue in a spatially-resolved manner. In some embodiments, the extension reaction can be performed separately from the sample handling apparatus described herein that is configured to perform the exemplary sandwiching process 104. The sandwich configuration of the sample 302, the first substrate (e.g., slide 303) and the second substrate (e.g., slide 304) may provide advantages over other methods of spatial analysis and/or analyte capture. For example, the sandwich configuration may reduce a burden of users to develop in house tissue sectioning and/or tissue mounting expertise. Further, the sandwich configuration may decouple sample preparation/tissue imaging from the barcoded array (e.g., spatially-barcoded capture probes 306) and enable selection of a particular region of interest of analysis (e.g., for a tissue section larger than the barcoded array). The sandwich configuration also beneficially enables spatial analysis without having to place a biological sample (e.g., tissue section) 302 directly on the second substrate (e.g., slide 304).

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism (also referred to herein as an adjustment mechanism) of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, wherein the permeabilization agent releases the analyte from the biological sample.

The sandwiching process methods described above can be implemented using a variety of hardware components. For example, the sandwiching process methods can be implemented using a sample holder (also referred to herein as a support device, a sample handling apparatus, and an array alignment device).

In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further includes an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane or the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

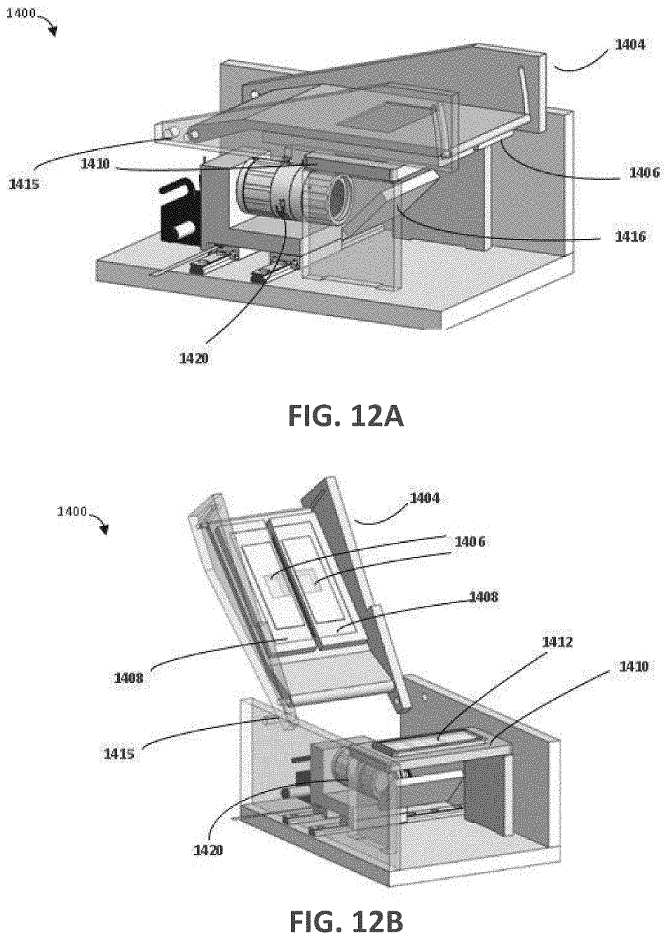

FIG. 12A is a perspective view of an example sample handling apparatus 1400 in a closed position in accordance with some example implementations. As shown, the sample handling apparatus 1400 includes a first member 1404, a second member 1410, optionally an image capture device 1420, a first substrate 1406, optionally a hinge 1415, and optionally a mirror 1416. The hinge 1415 may be configured to allow the first member 1404 to be positioned in an open or closed configuration by opening and/or closing the first member 1404 in a clamshell manner along the hinge 1415.

FIG. 12B is a perspective view of the example sample handling apparatus 1400 in an open position in accordance with some example implementations. As shown, the sample handling apparatus 1400 includes one or more first retaining mechanisms 1408 configured to retain one or more first substrates 1406. In the example of FIG. 12B, the first member 1404 is configured to retain two first substrates 1406; however, the first member 1404 may be configured to retain more or fewer first substrates 1406.

In some aspects, when the sample handling apparatus 1400 is in an open position (as in FIG. 12B), the first substrate 1406 and/or the second substrate 1412 may be loaded and positioned within the sample handling apparatus 1400 such as within the first member 1404 and the second member 1410, respectively. As noted, the hinge 1415 may allow the first member 1404 to close over the second member 1410 and form a sandwich configuration (e.g., the sandwich configuration shown in FIG. 11).

In some aspects, after the first member 1404 closes over the second member 1410, an adjustment mechanism (not shown) of the sample handling apparatus 1400 may actuate the first member 1404 and/or the second member 1410 to form the sandwich configuration for the permeabilization step (e.g., bringing the first substrate 1406 and the second substrate 1412 closer to each other and within a threshold distance for the sandwich configuration). The adjustment mechanism may be configured to control a speed, an angle, a force, or the like of the sandwich configuration.

In some embodiments, the biological sample (e.g., sample 302) may be aligned within the first member 1404 (e.g., via the first retaining mechanism 1408) prior to closing the first member 1404 such that a desired region of interest of the sample 302 is aligned with the barcoded array of the second substrate (e.g., the slide 304), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 1406 and/or the second substrate 1412 to maintain a minimum spacing between the first substrate 1406 and the second substrate 1412 during sandwiching. In some aspects, the permeabilization solution (e.g., permeabilization solution 305) may be applied to the first substrate 1406 and/or the second substrate 1412. The first member 1404 may then close over the second member 1410 and form the sandwich configuration. Analytes (e.g., protein and/or nucleic acid (e.g., DNA or RNA), such as mRNA transcripts) 308 may be captured by the capture probes 306 and may be processed for spatial analysis.

In some embodiments, during the permeabilization step, the image capture device 1420 may capture images of the overlap area (e.g., overlap area 710) between the tissue 302 and the capture probes 306. If more than one first substrates 1406 and/or second substrates 1412 are present within the sample handling apparatus 1400, the image capture device 1420 may be configured to capture one or more images of one or more overlap areas 710. Further details on support devices, sample holders, sample handling apparatuses, or systems for implementing a sandwiching process are described in, e.g., US. Patent Application Pub. No. 20210189475, and WO 2022/061152, each of which are incorporated by reference in their entirety.

Analytes within a biological sample may be released through disruption (e.g., permeabilization, digestion, etc.) of the biological sample or may be released without disruption. Various methods of permeabilizing (e.g., any of the permeabilization reagents and/or conditions described herein) a biological sample are described herein, including for example including the use of various detergents, buffers, proteases, and/or nucleases for different periods of time and at various temperatures. Additionally, various methods of delivering fluids (e.g., a buffer, a permeabilization solution) to a biological sample are described herein including the use of a substrate holder (e.g., for sandwich assembly, sandwich configuration, as described herein).

Provided herein are methods for delivering a fluid to a biological sample disposed on an area of a first substrate and an array disposed on a second substrate.

In some embodiments and with reference to FIG. 11, the sandwich configuration described herein between a first substrate comprising a biological sample (e.g., slide 303) and a second substrate comprising a spatially barcoded array (e.g., slide 304 with barcoded capture probes 306) may include a reagent medium (e.g., a liquid reagent medium, e.g., a permeabilization solution 305 or other target molecule release and capture solution) to fill a gap (e.g., gap 307). It may be desirable that the reagent medium be free from air bubbles between the slides to facilitate transfer of target molecules with spatial information. Additionally, air bubbles present between the slides may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104).

In some aspects, it may be possible to reduce or eliminate bubble formation between the slides using a variety of filling methods and/or closing methods.

Workflows described herein may include contacting a drop of the reagent medium (e.g., liquid reagent medium, e.g., a permeabilization solution 305) disposed on a first substrate or a second substrate with at least a portion of the second substrate or first substrate, respectively. In some embodiments, the contacting comprises bringing the two substrates into proximity such that the sample on the first substrate is aligned with the barcode array of capture probes on the second substrate.

In some embodiments, the drop includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures.

In the example sandwich maker workflows described herein, the reagent medium (e.g., liquid reagent medium, permeabilization solution 305) may fill a gap (e.g., the gap 307) between a first substrate (e.g., slide 303) and a second substrate (e.g., slide 304 with barcoded capture probes 306) to warrant or enable transfer of target molecules with spatial information. Described herein are examples of filling methods that may suppress bubble formation and suppress undesirable flow of transcripts and/or target molecules or analytes. Robust fluidics in the sandwich making described herein may preserve spatial information by reducing or preventing deflection of molecules as they move from the tissue slide to the capture slide.

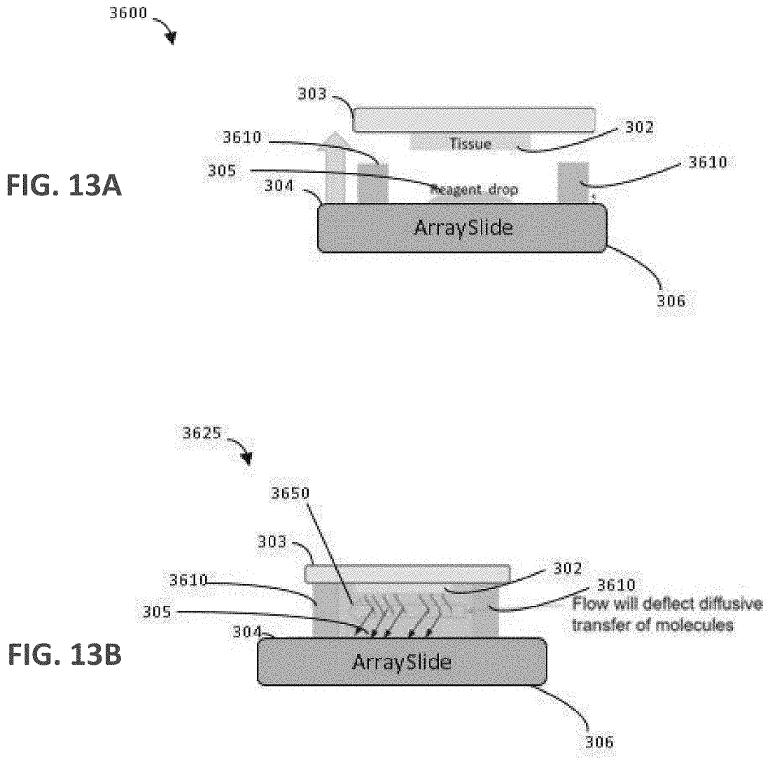

FIG. 13A shows an exemplary sandwiching process 3600 where a first substrate (e.g., slide 303), including a biological sample 302 (e.g., a tissue section), and a second substrate (e.g., slide 304 including spatially barcoded capture probes 306) are brought into proximity with one another. As shown in FIG. 13A, a liquid reagent drop (e.g., permeabilization solution 305) is introduced on the second substrate in proximity to the capture probes 306 and in between the biological sample 302 and the second substrate (e.g., slide 304 including spatially barcoded capture probes 306). The permeabilization solution 305 may release analytes that can be captured by the capture probes 306 of the array. As further shown, one or more spacers 3610 may be positioned between the first substrate (e.g., slide 303) and the second substrate (e.g., slide 304 including spatially barcoded capture probes 306). The one or more spacers 3610 may be configured to maintain a separation distance between the first substrate and the second substrate. While the one or more spacers 3610 is shown as disposed on the second substrate, the spacer may additionally or alternatively be disposed on the first substrate.

In some embodiments, the one or more spacers 3610 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 13B shows a fully formed sandwich configuration creating a chamber 3650 formed from the one or more spacers 3610, the first substrate (e.g., the slide 303), and the second substrate (e.g., the slide 304 including spatially barcoded capture probes 306) in accordance with some example implementations. In the example of FIG. 13B, the liquid reagent (e.g., the permeabilization solution 305) fills the volume of the chamber 3650 and may create a permeabilization buffer that allows analytes (e.g., mRNA transcripts and/or other molecules) to diffuse from the biological sample 302 toward the capture probes 306 of the second substrate (e.g., slide 304). In some aspects, flow of the permeabilization buffer may deflect transcripts and/or molecules from the biological sample 302 and may affect diffusive transfer of analytes for spatial analysis. A partially or fully sealed chamber 3650 resulting from the one or more spacers 3610, the first substrate, and the second substrate may reduce or prevent flow from undesirable convective movement of transcripts and/or molecules over the diffusive transfer from the biological sample 302 to the capture probes 306.

In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

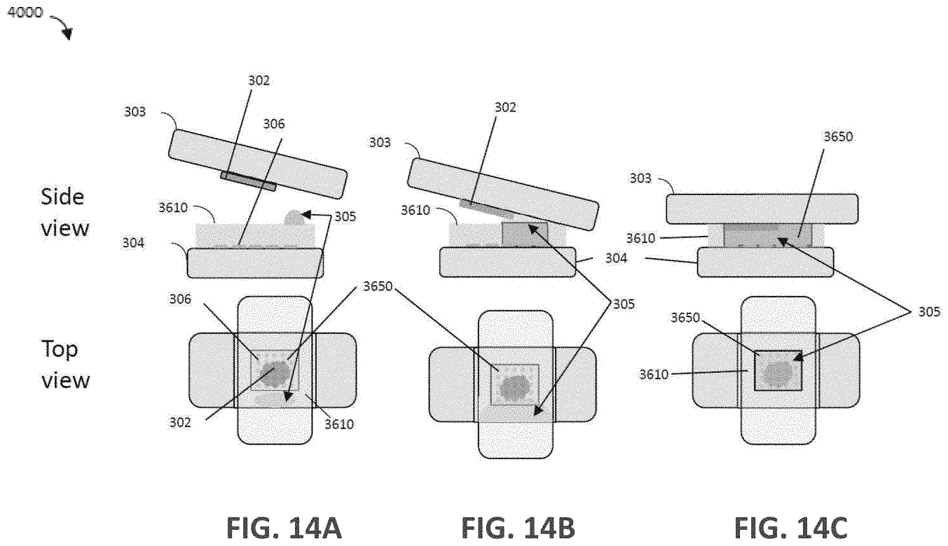

FIGS. 14A-14C depict a side view and a top view of an exemplary angled closure workflow 4000 for sandwiching a first substrate (e.g., slide 303) having a biological sample 302 and a second substrate (e.g., slide 304 having capture probes 306) in accordance with some example implementations.

FIG. 14A depicts the first substrate (e.g., the slide 303 including biological sample 302) angled over (superior to) the second substrate (e.g., slide 304). As shown, a drop of the reagent medium (e.g., permeabilization solution) 305 is located on the spacer 3610 toward the right-hand side of the side view in FIG. 14A. While FIG. 14A depicts the reagent medium on the right hand side of side view, it should be understood that such depiction is not meant to be limiting as to the location of the reagent medium on the spacer.

FIG. 14B shows that as the first substrate lowers, and/or as the second substrate rises, the dropped side of the first substrate (e.g., a side of the slide 303 angled toward the second substrate) may contact the drop of the reagent medium 305. The dropped side of the first substrate may urge the reagent medium 305 toward the opposite direction (e.g., towards an opposite side of the spacer 3610, towards an opposite side of the first substrate relative to the dropped side). For example, in the side view of FIG. 14B, the reagent medium 305 may be urged from right to left as the sandwich is formed.

In some embodiments, the first substrate and/or the second substrate are further moved to achieve an approximately parallel arrangement of the first substrate and the second substrate.

FIG. 14C depicts a full closure of the sandwich between the first substrate and the second substrate with the spacer 3610 contacting both the first substrate and the second substrate and maintaining a separation distance and optionally the approximately parallel arrangement between the two substrates. As shown in the top view of FIG. 14C, the spacer 3610 fully encloses and surrounds the biological sample 302 and the capture probes 306, and the spacer 3610 forms the sides of chamber 3650 which holds a volume of the reagent medium 305.

While FIGS. 14A-14C depict the first substrate (e.g., the slide 303 including biological sample 302) angled over (superior to) the second substrate (e.g., slide 304) and the second substrate comprising the spacer 3610, it should be understood that an exemplary angled closure workflow can include the second substrate angled over (superior to) the first substrate and the first substrate comprising the spacer 3610.

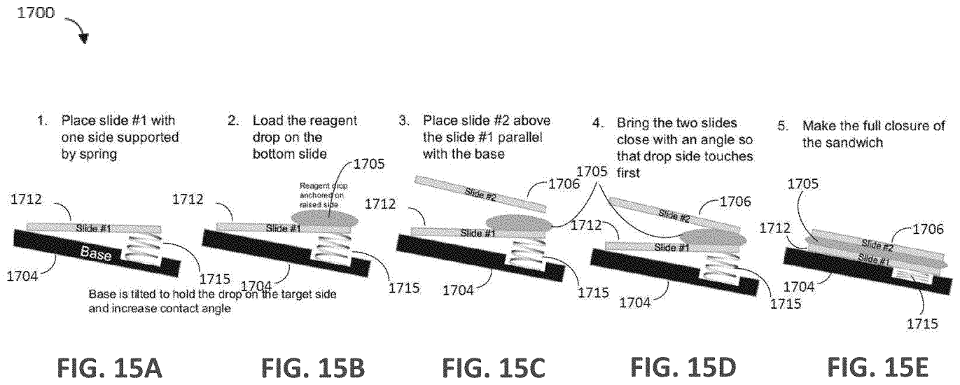

FIGS. 15A-15E depict an example workflow 1700 for an angled sandwich assembly in accordance with some example implementations. As shown in FIG. 15A, a substrate 1712 (e.g., a first substrate such as slide 303 or a second substrate such as slide 304 comprising spatially barcoded capture probes 306) may be positioned and placed on a base 1704 (e.g., a first member or a second member of a sample holder disclosed herein) with a side of the substrate 1712 supported by a spring 1715. The spring 1715 may extend from the base 1704 in a superior direction and may be configured to dispose the substrate 1712 along a plane angled differently than the base 1704. The angle of the substrate 1712 may be such that a drop of reagent medium 1705 (e.g., drop of liquid reagent medium) placed on the surface of the substrate 1712 (e.g., a surface of a spacer attached to the substrate) will not fall off the surface (e.g., due to gravity). The angle may be determined based on a gravitational force versus any surface force to move the drop away from and off the substrate 1712.

FIG. 15B depicts a drop 1705 of reagent medium placed on the substrate 1712. As shown, the drop 1705 is located on the side of the substrate 1712 contacting the spring 1715 and is located in proximity and above (superior to) the spring 1715.

As shown in FIG. 15C, another substrate 1706 may be positioned above (superior to) the substrate 1712 and at an angle substantially parallel with the base 1704. For example, in cases wherein substrate 1712 is a second substrate disclosed herein (e.g., slide 304 comprising spatially barcoded capture probes), substrate 1706 may be a first substrate disclosed herein (e.g., slide 303). In cases wherein substrate 1712 is a first substrate disclosed herein (e.g., slide 303), substrate 1706 may be a second substrate (e.g., slide 304 comprising spatially barcoded capture probes). In some cases, another base (not shown) supporting substrate 1706 (e.g., a first member or a second member of a sample holder disclosed herein) may be configured to retain substrate 1706 at the angle substantially parallel to the base 1704.

As shown in FIG. 15D, substrate 1706 may be lowered toward the substrate 1712 such that a dropped side of the substrate 1706 contacts the drop 1705 first. In some aspects, the dropped side of the substrate 1706 may urge the drop 1705 toward the opposite side of the substrate 1706. In some embodiments, the substrate 1712 may be moved upward toward the substrate 1706 to accomplish the contacting of the dropped side of the substrate 1706 with the drop 1705.

FIG. 15E depicts a full sandwich closure of the substrate 1706 and the substrate 1712 with the drop of reagent medium 1705 positioned between the two sides. In some aspects and as shown, as the substrate 1706 is lowered onto the drop 1705 and toward the substrate 1712 (and/or as the substrate 1712 is raised up toward the substrate 1706), the spring 1715 may compress and the substrate 1712 may lower to the base 1704 and become substantially parallel with the substrate 1706.

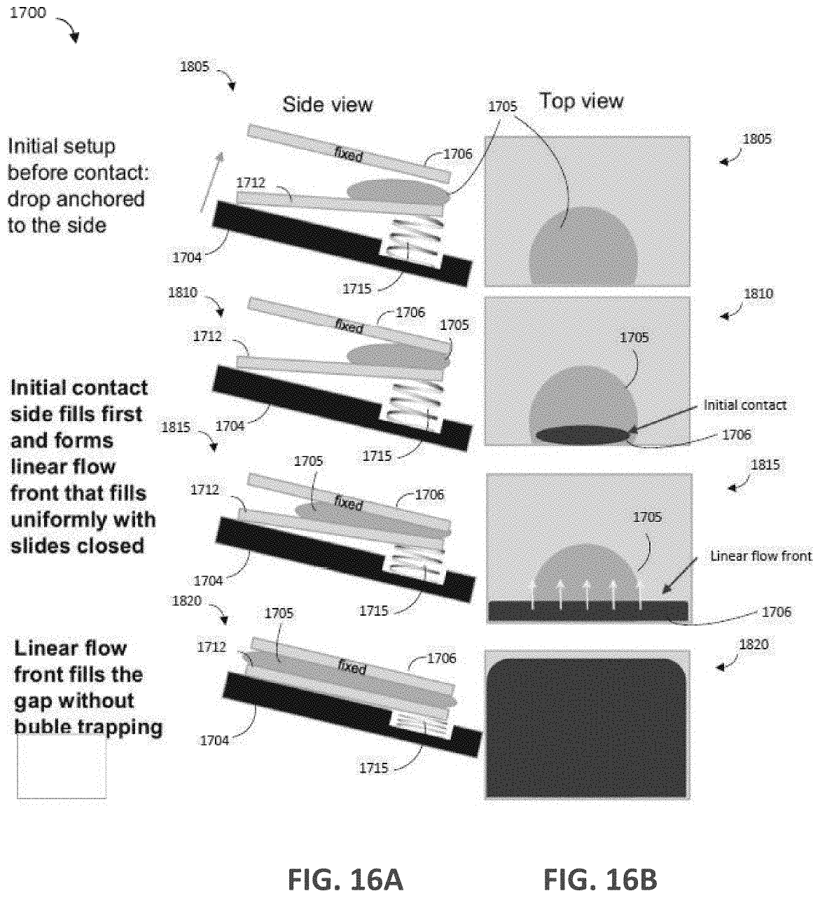

FIG. 16A is a side view of the angled closure workflow 1700 in accordance with some example implementations. FIG. 16B is a top view of the angled closure workflow 1700 in accordance with some example implementations. As shown at 1805 and in accordance with FIGS. 15C-15D, the drop of reagent medium 1705 is positioned to the side of the substrate 1712 contacting the spring 1715.

At step 1810, the dropped side of the angled substrate 1706 contacts the drop of reagent medium 1705 first. The contact of the substrate 1706 with the drop of reagent medium 1705 may form a linear or low curvature flow front that fills uniformly with the slides closed.

At step 1815, the substrate 1706 is further lowered toward the substrate 1712 (or the substrate 1712 is raised up toward the substrate 1706) and the dropped side of the substrate 1706 may contact and may urge the liquid reagent toward the side opposite the dropped side and creating a linear or low curvature flow front that may prevent or reduce bubble trapping between the slides. As further shown, the spring 1715 may begin to compress as the substrate 1706 is lowered.

At step 1820, the drop of reagent medium 1705 fills the gap (e.g., the gap 307) between the substrate 1706 and the substrate 1712. The linear flow front of the liquid reagent may form by squeezing the drop 1705 volume along the contact side of the substrate 1712 and/or the substrate 1706. Additionally, capillary flow may also contribute to filling the gap area. As further shown in step 1820, the spring 1715 may be fully compressed such that the substrate 1706, the substrate 1712, and the base 1704 are substantially parallel to each other.

In some aspects, an angled closure workflow disclosed herein (e.g., FIGS. 14A-14C, 15A-15E, 16A-16B) may be performed by a sample handling apparatus (e.g., as described in WO 2022/061152, which is hereby incorporated by reference in its entirety). Further details on angled closure workflows, and devices and systems for implementing an angled closure workflow, are described in WO 2021/252747 and WO 2022/061152, which are hereby incorporated by reference in their entirety.

Additional configurations for reducing or eliminating bubble formation, and/or for reducing unwanted fluid flow, are described in WO 2021/252747, which is hereby incorporated by reference in its entirety.

II. Resolving Secondary Structures with Stretching Moieties

A. Introduction

Disclosed herein are spatial methods to enhance detection of analytes on an array. It has been found that the analyte (e.g., a nucleic acid analyte, such as mRNA) can form secondary structures, either naturally or during capture of an analyte (e.g., an mRNA analyte) via hybridization to a capture domain in a capture probe, thereby affecting efficiency of analyte detection in at least two ways. First, secondary structures can sterically prevent additional analytes from hybridizing to nearby probes, leading to fewer analytes captured on the array. Second, secondary structures can affect the efficiency of replication of the capture probe and analyte, thereby lessening the number of analytes that could be captured on an array. The methods disclosed herein result in resolution (e.g., stretching) of the analyte to uncoil the secondary structures to allow for efficient capture and/or replication on a spatial array.

Featured herein are methods for determining the presence or abundance of an analyte in a biological sample. In some instances, the methods include providing a biological sample on a substrate comprising a plurality of capture probes, wherein a capture probe of the plurality of capture probes comprises a capture domain; releasing the analyte from the biological sample; affixing a stretching moiety to the analyte; hybridizing the analyte to the capture domain of the capture probe; applying a stretching force to the stretching moiety, thereby elongating the analyte hybridized to the capture domain; and generating an extended capture probe using the analyte as a template. In some instances, the steps of releasing the analyte and affixing the stretching moiety to the analyte are performed at the same time. In some instances, the step of releasing the analyte occurs before the step of affixing the stretching moiety to the analyte. In some instances, the step of releasing the analyte occurs after the step of affixing the stretching moiety to the analyte.

In some instances, the methods include providing a biological sample on a substrate comprising a plurality of capture probes, wherein a capture probe of the plurality of capture probes comprises a capture domain; releasing the analyte from the biological sample; affixing a stretching moiety to the analyte; hybridizing the analyte to the capture domain of the capture probe; applying a stretching force to the stretching moiety, thereby elongating the analyte hybridized to the capture domain; hybridizing a padlock oligonucleotide to a target analyte hybridized to a capture domain such that the padlock oligonucleotide is circularized, wherein the padlock oligonucleotide comprises: (i) a first sequence that is substantially complementary to a first portion of the analyte, or a complement thereof; (ii) a backbone sequence, and (iii) a second sequence that is substantially complementary to a second portion of the analyte, or a complement thereof; ligating the first sequence to the second sequence of the padlock oligonucleotide, thereby generating a circularized padlock oligonucleotide; amplifying the circularized padlock oligonucleotide thereby creating an amplified circularized padlock oligonucleotide, and identifying the presence or abundance of the analyte in the biological sample.

In some instances, the methods include providing the biological sample on a first substrate; aligning the first substrate with a second substrate comprising an array, such that at least a portion of the biological sample is aligned with at least a portion of the array, where the array comprises a plurality of capture probes, where a capture probe of the plurality of capture probes comprises a spatial barcode and a capture domain; releasing the nucleic acid analyte from the biological sample, such that the nucleic acid analyte actively or passively migrates toward the capture probe, and binds the capture probe; affixing a stretching moiety to the nucleic acid analyte; hybridizing the nucleic acid analyte to the capture domain; applying a stretching force to the stretching moiety, thereby elongating the nucleic acid analyte hybridized to the capture domain; and extending the capture probe using the nucleic acid analyte as a template, thereby generating an extended capture probe.

The present disclosure also provides additional method embodiments as well as compositions, systems, and kits described herein.

B. Attaching a Stretching Moiety to an Analyte

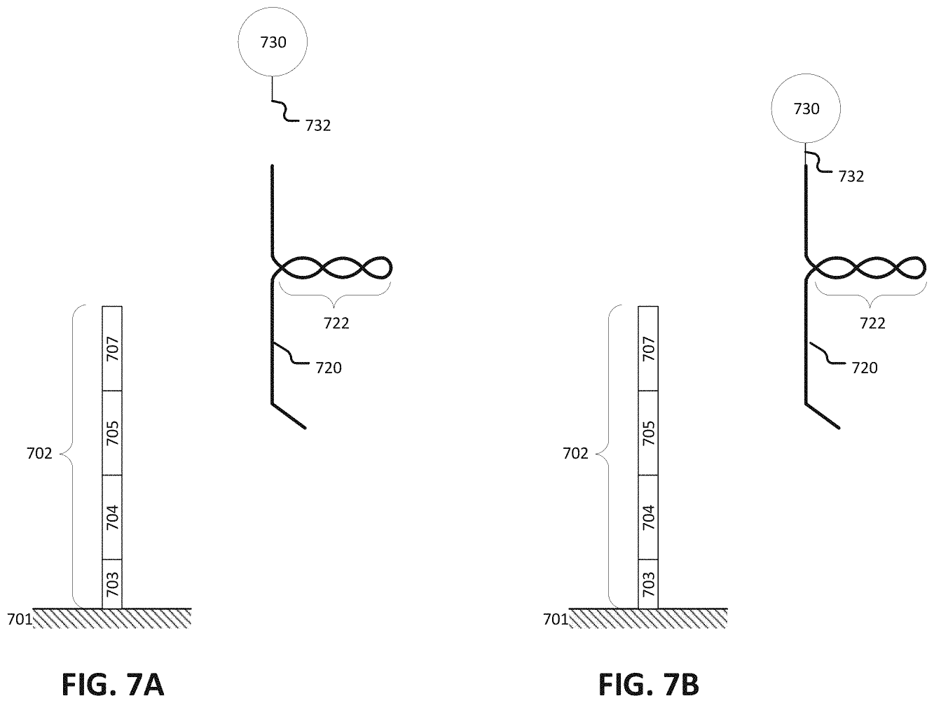

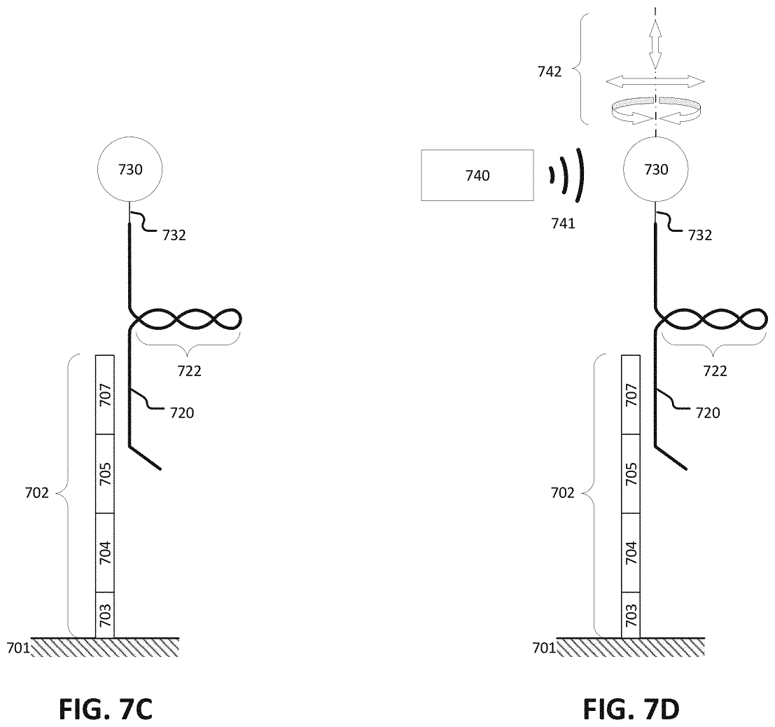

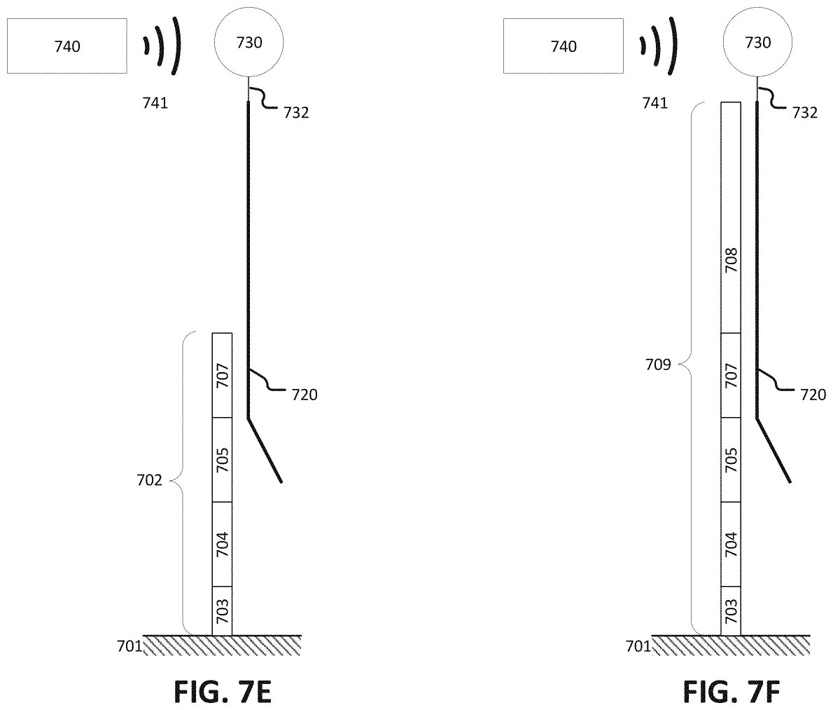

In some embodiments, the methods provided herein increase analyte detection by attaching a stretching moiety to a target analyte. As shown in FIG. 7E and FIG. 7F, after the target analyte 720 is bound to the capture domain 707 of the capture probe 702, a reverse transcriptase enzyme can add a sequence 708 to the 3′ end of the capture probe that is complementary to the target analyte sequence 720, to generate an extended capture probe 709. An exemplary workflow for the creation of an extended capture probe is shown in FIG. 7A through FIG. 7F. FIG. 7A depicts a capture probe 702 comprising a cleavage domain 703, functional sequence 704, spatial barcode 705, and a capture domain 707 immobilized on a substrate 701. The functional sequence 704 can be any of the exemplary functional sequences described herein. The spatial barcode 705 can be any spatial barcode as described herein. The capture domain 707 can include a sequence that specifically hybridizes to a target analyte. The target analyte 720 shown in FIG. 7A is depicted as an mRNA target analyte, but the analyte 720 can be any target analyte or analyte capture sequence as described herein.

Referring to FIG. 7A, the target analyte 720 includes a secondary structure 722. The secondary structure 722 can occlude a portion of the total length of the target analyte 720, such as occluding a portion of the target analyte 720 from binding to the capture domain 707, or binding to a reverse transcriptase. Examples of secondary structure 722 can include, but are not limited to coiling, stem-loops, pseudo-knots, or alternative helix structures (e.g., A-, of Z-form helix structures). The secondary structure 722 can occlude a portion of the target analyte 720 total length in a range from 5% to 90% (e.g., 10% to 90%, 20% to 90%, 30% to 90%, 40% to 90%, 50% to 90%, 60% to 90%, 70% to 90%, 80% to 90%, 5% to 10%, 5% to 20%, 5% to 30%, 5% to 40%, 5% to 50%, 5% to 60%, 5% to 70%, 5% to 80%, 10% to 70%, 20% to 60%, or 30% to 50%).

FIG. 7A includes a stretching moiety 730. The stretching moiety 730 affixes to one end of the target analyte 720 through a linkage site 732. The linkage site 732 provides a permanent or temporary (e.g., reversible) connection between the stretching moiety 730 and the target analyte 720. The linkage site 732 can be attached to the stretching moiety 730, or be attached to the target analyte 720. In some embodiments, the target analyte 720 is an oligonucleotide including a 5′ cap. A 5′ cap is an altered nucleotide on the 5′ end of a target analyte, such as an mRNA. For example, an mRNA 5′ cap can be a guanosine nucleotide. The guanosine nucleotide can be modified enzymatically and/or replaced by a biotinylated nucleotide or a digoxigenin nucleotide to create the altered nucleotide 5′ cap. In some embodiments, the linkage site 732 is attached to the altered nucleotide 5′ cap of the target analyte 720.

In some embodiments, the target analyte 720 includes a linkage site 732 on the 3′ end. For example, mRNA includes a poly-A tail on the 3′ end of the target analyte 720. A tailing polymerase, or terminal transferase, can introduce a biotinylated nucleotide or digoxigenin nucleotide to the 3′ end of the target analyte 720.

In some embodiments, the target analyte 720 includes a linkage site 732 which is a nucleotide sequence. In such embodiments, the stretching moiety 730 includes a nucleotide sequence complementary to the linkage site 732 nucleotide sequence. The complementary nucleotide sequences bind, thereby attaching the stretching moiety 730 to the linkage site 732.

Referring to FIG. 7B, in some embodiments, the linkage site 732 includes one half of a binding pair. For example, the target analyte 720 can include a cap at one end including a first half of a binding pair. The linkage site 732 affixed to the stretching moiety 730 can include a second half of the binding pair, which specifically binds to the first half. Examples of a binding pair can include a biotin-streptavidin pair, an antibody-target pair (e.g., digoxigenin-anti-digoxigenin), or a protein-ligand pair (e.g., biotin-avidin, or biotin-streptavidin). For example, the linkage site 732 can include a biotin moiety and the target analyte 720 can include an avidin moiety, or vice versa. In some embodiments, the linkage site 732 can be referred to as a binding moiety and a binding pair can be referred to as a first binding moiety and a second binding moiety. For examine, the biotin moiety can be the first binding moiety and the avidin moiety can be the second binding moiety. In some embodiments, the first binding moiety can be the linkage site 732 and the target analyte 720 can include the second binding moiety, or vice versa. The binding moiety affixed to the target analyte 720 can be affixed to the 5′ end or the 3′ end. In some instances, the binding moiety affixed to the target analyte 720 is affixed to the 5′ end. In some instances, the binding moiety affixed to the target analyte 720 is affixed to the 3′ end. It is appreciated that one skilled in the art could refer to the moiety associated with the stretching moiety as the second binding moiety instead of the first binding moiety, and vice versa. It should be readily apparent to one skilled in the art to which binding moiety is referred throughout the specification

FIG. 7B shows the stretching moiety 730 affixed to the target analyte 720 via linkage site 732 and FIG. 7C shows the target analyte 720 including the affixed stretching moiety 730 hybridized to the capture domain 707 of the capture probe 702. In some embodiments, the linkage site 732 can include one or more cleavage moieties. Examples of cleavage moieties can include uracils, a disulfide linker, photocleavable modified nucleotides, or any cleavage domain 707 as described herein.

In some embodiments, the linkage site 732 comprises a cleavage domain capable of cleavage by an enzyme. An enzyme can be added to cleave the cleavage domain, resulting in release of the capture probe from the feature. As another example, heating can also result in degradation of the cleavage domain and release of the attached capture probe from the array feature. In some embodiments, laser radiation is used to heat and degrade cleavage domains at specific locations. In some embodiments, the cleavage domain is a photo-sensitive chemical bond (e.g., a chemical bond that dissociates when exposed to light such as ultraviolet light). In some embodiments, the cleavage domain can be an ultrasonic cleavage domain. For example, ultrasonic cleavage can depend on nucleotide sequence, length, pH, ionic strength, temperature, and the ultrasonic frequency (e.g., 22 kHz, 44 kHz) (Grokhovsky, S. L., Specificity of DNA cleavage by ultrasound, Molecular Biology, 40(2), 276-283 (2006)).

Additional linkers that can be utilized at the linkage site include linkers described in WO 2020/176788 and/or U.S. Patent Application Publication No. 2020/0277663, each of which is incorporated by reference in its entirety.

Referring to FIG. 7C, the stretching moiety 730 is a moiety responsive to an applied field. For example, the stretching moiety can respond to an electromagnetic field. In some embodiments, the stretching moiety 730 is responsive to a magnetic field, an electric field, or a light field. As an example, the stretching moiety 730 can be a polystyrene moiety responsive to a directed light field, e.g., optical tweezers. Alternatively, the stretching moiety 730 can be composed of a magnetic material responsive to a magnetic field (e.g., a magnetic moiety, magnetic bead). For example, the stretching moiety 730 can be composed of a magnetic material (e.g., neodymium), a paramagnetic material (e.g., aluminum, gold, copper), or a non-magnetic material (e.g., a polymer material) coated in or impregnated with a magnetic or paramagnetic material.

Referring to FIG. 7D, a field application instrument 740 (e.g., a magnetic instrument; e.g., a set of magnetic tweezers) applies a field to the environment of the stretching moiety 730, target analyte 720, and capture probe 702. The field application instrument 740 can apply an electric field, a magnetic field, or a light field to the stretching moiety 730. Examples of field application instrument 740 can include permanent magnets, or electromagnets. The field application instrument 740 can include control software to vary the strength and direction of the applied field 741 and thereby the stretching force 742. The applied field 741 creates a stretching force 742 on the stretching moiety 730 in at least one direction. For example, the stretching force 742 can be a force having a magnitude, such as a linear force orthogonal to a plane of an upper surface of the substrate 701, a rotational force around a rotational axis orthogonal to the plane of the upper surface of the substrate 701, or both. The magnitude of the stretching force 742 can be in the range from 0.05 piconewtons (pN) to 100 pN (e.g., 0.1 pN to 100 pN, 1 pN to 100 pN, 10 pN to 100 pN, 20 pN to 100 pN, 40 pN to 100 pN, 50 pN to 100 pN, 60 pN to 100 pN, 80 pN to 100 pN, 0.05 pN to 80 pN, 0.05 pN to 60 pN, 0.05 pN to 50 pN, 0.05 pN to 40 pN, 0.05 pN to 20 pN, 0.05 pN to 10 pN, 0.05 pN to 1 pN, 0.05 pN to 0.1 pN, 0.1 pN to 80 pN, 1 to 50 pN, 10 pN to 20 pN, 0.1 pN to 0.5 pN, or 0.2 pN to 0.4 pN).). The rotational force around a rotational axis orthogonal to the plane of the upper surface of the substrate 701 can be in a clockwise, or anti-clockwise radial direction around the rotational axis.

In some embodiments, the stretching force 742 is applied constantly, e.g., a constant force. In some embodiments, the stretching force 742 is modulated, e.g., changes. In some embodiments, the stretching force 742 is modulated in a pattern, e.g., a non-constant force, or a cyclic pattern between a maximum and a minimum. For example, the stretching force 742 is applied for a first time period at a first magnitude in a first direction. The stretching force 742 is then optionally applied for a second or more time period at a second or more magnitude in a second or more direction. For example, the stretching force 742 is applied in a range from about 1 second (s) to about 10 minutes (min), about 30 s to about 5 min, or about 1 min to about 3 min. The stretching force 742 can be modulated, e.g., changed, more than one time or for more than one time period (e.g., more than two times, more than three times, more than five times, more than ten times, more than two time periods, more than three time periods, more than five time periods, or more than ten time periods).

Referring to FIG. 7E, the stretching force 742 created by the applied field 741 between the capture probe 702 affixed to the substrate 701 and the stretching moiety 730 translates along the backbone of the target analyte 720 thereby eliminating the secondary structure 722 and elongating the target analyte 720. FIG. 7E shows the elongated target analyte 720 without the secondary structure 722 and the field application instrument 740 applying the applied field 741 to the stretching moiety 730 thereby stretching the target analyte 720 to a length orthogonal to the plane of the upper surface of the substrate 701. In some embodiments, the target analyte 720 is stretched to a maximum linear length orthogonal to the plane of the upper surface of the substrate 701. In some embodiments, the target analyte 720 is stretched to a portion of the maximum linear length.

As shown in FIG. 7F, the target analyte 720 is hybridized to the capture domain 707 of the capture probe 702 and a reverse transcriptase enzyme can add a sequence 708 to the 3′ end of the capture probe that is complementary to a sequence of the target analyte 720, to generate an extended capture probe 709 using the target analyte 720 as the template. The extended capture probe 709 can be released from the substrate 701 and the target analyte 720 released from the extended capture probe 709. Following the release of the extended capture probe 709 from the substrate, the solution containing the capture probe 709 can be transferred to a fresh container and optionally neutralized before being used to generate a library for analyte capture determinations (e.g., using any of the exemplary methods described herein). In another embodiment, the hybridized target analyte with the affixed stretching moiety is released from the extended capture probe and second strand DNA is synthesized (not shown) from the extended capture probe. In this scenario, the second strand DNA, which includes a complement of the extended capture probe 709, is released from the extended capture probe, transferred, a sequencing library is generated and sequenced for spatial analysis.

C. Capture Probes

The disclosure provides capture probes affixed to an array. In some instances, each capture probe includes at least one capture domain. The “capture domain” can be an oligonucleotide, a polypeptide, a small molecule, or any combination thereof, that binds specifically to a desired analyte. In some embodiments, a capture domain can be used to capture or detect a desired analyte.

In some embodiments, the capture domain is a functional nucleic acid sequence configured to interact with one or more analytes, such as one or more different types of nucleic acids (e.g., RNA molecules and DNA molecules). In some embodiments, the functional nucleic acid sequence can include an N-mer sequence (e.g., a random N-mer sequence), which N-mer sequences are configured to interact with a plurality of DNA molecules. In some embodiments, the functional sequence can include a poly(T) sequence, which poly(T) sequences are configured to interact with messenger RNA (mRNA) molecules via the poly(A) tail of an mRNA transcript. In some embodiments, the functional nucleic acid sequence is the binding target of a protein (e.g., a transcription factor, a DNA binding protein, or a RNA binding protein), where the analyte of interest is a protein. In some embodiments, the functional sequence can be a known sequence for interacting with another known sequence, for example in a mRNA or DNA molecule.

Capture probes can include ribonucleotides and/or deoxyribonucleotides as well as synthetic nucleotide residues that are capable of participating in Watson-Crick type or analogous base pair interactions. In some embodiments, the capture domain is capable of priming a reverse transcription reaction to generate cDNA that is complementary to the captured RNA molecules. In some embodiments, the capture domain of the capture probe can prime a DNA extension (polymerase) reaction to generate DNA that is complementary to the captured DNA molecules. In some embodiments, the capture domain can template a ligation reaction between the captured DNA molecules and a surface probe that is directly or indirectly immobilized on the substrate. In some embodiments, the capture domain can be ligated to one strand of the captured DNA molecules. For example, disclosed herein are a PBCV-1 ligase, a Chlorella virus ligase (each sometimes referred to as a SplintR ligase). PBCV-1 ligase or a Chlorella virus ligase along with RNA or DNA sequences (e.g., degenerate RNA) can be used to ligate a single-stranded DNA or RNA to the capture domain. In some embodiments, ligases with RNA-templated ligase activity, e.g., PBCV-1 ligase, a Chlorella virus ligase, T4 RNA ligase 2 or KOD ligase, can be used to ligate a single-stranded DNA or RNA to the capture domain. In some embodiments, a capture domain includes a splint oligonucleotide. In some embodiments, a capture domain captures a splint oligonucleotide.

In some embodiments, the capture domain is located at the 3′ end of the capture probe and includes a free 3′ end that can be extended, e.g., by template dependent polymerization, to form an extended capture probe as described herein. In some embodiments, the capture domain includes a nucleotide sequence that is capable of hybridizing to nucleic acid, e.g., RNA or other analyte, present in the cells of the biological sample contacted with the array. In some embodiments, the capture domain can be selected or designed to bind selectively or specifically to a target nucleic acid. For example, the capture domain can be selected or designed to capture mRNA by way of hybridization to the mRNA poly(A) tail. Thus, in some embodiments, the capture domain includes a poly(T) DNA oligonucleotide, e.g., a series of consecutive deoxythymidine residues linked by phosphodiester bonds, which is capable of hybridizing to the poly(A) tail of mRNA. In some embodiments, the capture domain can include nucleotides that are functionally or structurally analogous to a poly(T) tail. For example, a poly(U) oligonucleotide or an oligonucleotide included of deoxythymidine analogues. In some embodiments, the capture domain includes at least 10, 11, 12, 13, 14, 15, 16, 17, 18, 19, or 20 nucleotides. In some embodiments, the capture domain includes at least 25, 30, or 35 nucleotides.

In some embodiments, a capture probe includes a capture domain having a sequence that is capable of binding to mRNA and/or genomic DNA. For example, the capture probe can include a capture domain that includes a nucleic acid sequence (e.g., a poly(T) sequence) capable of binding to a poly(A) tail of an mRNA and/or to a poly(A) homopolymeric sequence present in genomic DNA. In some embodiments, a homopolymeric sequence is added to an mRNA molecule or a genomic DNA molecule using a terminal transferase enzyme in order to produce an analyte that has a poly(A) or poly(T) sequence. For example, a poly(A) sequence can be added to an analyte (e.g., a fragment of genomic DNA) thereby making the analyte capable of capture by a poly(T) capture domain.

In some embodiments, random sequences, e.g., random hexamers or similar sequences, can be used to form all or a part of the capture domain. For example, random sequences can be used in conjunction with poly(T) (or poly(T) analogue) sequences. Thus, where a capture domain includes a poly(T) (or a “poly(T)-like”) oligonucleotide, it can also include a random oligonucleotide sequence (e.g., “poly(T)-random sequence” probe). This can, for example, be located 5′ or 3′ of the poly(T) sequence, e.g., at the 3′ end of the capture domain. The poly(T)-random sequence probe can facilitate the capture of the mRNA poly(A) tail. In some embodiments, the capture domain can be an entirely random sequence. In some embodiments, degenerate capture domains can be used.

In some embodiments, a pool of two or more capture probes form a mixture, where the capture domain of one or more capture probes includes a poly(T) sequence and the capture domain of one or more capture probes includes random sequences. In some embodiments, a pool of two or more capture probes form a mixture where the capture domain of one or more capture probes includes poly(T)-like sequence and the capture domain of one or more capture probes includes random sequences. In some embodiments, a pool of two or more capture probes form a mixture where the capture domain of one or more capture probes includes a poly(T)-random sequences and the capture domain of one or more capture probes includes random sequences. In some embodiments, probes with degenerate capture domains can be added to any of the preceding combinations listed herein. In some embodiments, probes with degenerate capture domains can be substituted for one of the probes in each of the pairs described herein.

The capture domain can be based on a particular gene sequence or particular motif sequence or common/conserved sequence, that it is designed to capture (i.e., a sequence-specific capture domain). Thus, in some embodiments, the capture domain is capable of binding selectively to a desired sub-type or subset of nucleic acid, for example a particular type of RNA, such as mRNA, rRNA, tRNA, SRP RNA, tmRNA, snRNA, snoRNA, SmY RNA, scaRNA, gRNA, RNase P, RNase MRP, TERC, SL RNA, aRNA, cis-NAT, crRNA, lncRNA, miRNA, piRNA, siRNA, shRNA, tasiRNA, rasiRNA, 7SK, eRNA, ncRNA or other types of RNA. In a non-limiting example, the capture domain can be capable of binding selectively to a desired subset of ribonucleic acids, for example, microbiome RNA, such as 16S rRNA.

In some embodiments, a capture domain includes an “anchor” or “anchoring sequence”, which is a sequence of nucleotides that is designed to ensure that the capture domain hybridizes to the intended analyte. In some embodiments, an anchor sequence includes a sequence of nucleotides, including a 1-mer, 2-mer, 3-mer or longer sequence. In some embodiments, the short sequence is random. For example, a capture domain including a poly(T) sequence can be designed to capture an mRNA. In such embodiments, an anchoring sequence can include a random 3-mer (e.g., GGG) that helps ensure that the poly(T) capture domain hybridizes to an mRNA. In some embodiments, an anchoring sequence can be VN, N, or NN. Alternatively, the sequence can be designed using a specific sequence of nucleotides. In some embodiments, the anchor sequence is at the 3′ end of the capture domain. In some embodiments, the anchor sequence is at the 5′ end of the capture domain.

In some embodiments, capture domains of capture probes are blocked prior to contacting the biological sample with the array, and blocking probes are used when the nucleic acid in the biological sample is modified prior to its capture on the array. In some embodiments, the blocking probe is used to block or modify the free 3′ end of the capture domain. In some embodiments, blocking probes can be hybridized to the capture probes to mask the free 3′ end of the capture domain, e.g., hairpin probes, partially double stranded probes, or complementary sequences. In some embodiments, the free 3′ end of the capture domain can be blocked by chemical modification, e.g., addition of an azidomethyl group as a chemically reversible capping moiety such that the capture probes do not include a free 3′ end. Blocking or modifying the capture probes, particularly at the free 3′ end of the capture domain, prior to contacting the biological sample with the array, prevents modification of the capture probes, e.g., prevents the addition of a poly(A) tail to the free 3′ end of the capture probes.

Non-limiting examples of 3′ modifications include dideoxy C-3′ (3′-ddC), 3′ inverted dT, 3′ C3 spacer, 3′Amino, and 3′ phosphorylation. In some embodiments, the nucleic acid in the biological sample can be modified such that it can be captured by the capture domain. For example, an adaptor sequence (including a binding domain capable of binding to the capture domain of the capture probe) can be added to the end of the nucleic acid, e.g., fragmented genomic DNA. In some embodiments, this is achieved by ligation of the adaptor sequence or extension of the nucleic acid. In some embodiments, an enzyme is used to incorporate additional nucleotides at the end of the nucleic acid sequence, e.g., a poly(A) tail. In some embodiments, the capture probes can be reversibly masked or modified such that the capture domain of the capture probe does not include a free 3′ end. In some embodiments, the 3′ end is removed, modified, or made inaccessible so that the capture domain is not susceptible to the process used to modify the nucleic acid of the biological sample, e.g., ligation or extension.

In some embodiments, the capture domain of the capture probe is modified to allow the removal of any modifications of the capture probe that occur during modification of the nucleic acid molecules of the biological sample. In some embodiments, the capture probes can include an additional sequence downstream of the capture domain, e.g., 3′ to the capture domain, namely a blocking domain.

In some embodiments, the capture domain of the capture probe can be a non-nucleic acid domain. Examples of suitable capture domains that are not exclusively nucleic-acid based include, but are not limited to, proteins, peptides, aptamers, antigens, antibodies, and molecular analogs that mimic the functionality of any of the capture domains described herein.

Each capture probe can optionally include at least one cleavage domain. The cleavage domain represents the portion of the probe that is used to reversibly attach the probe to an array feature, as will be described further herein. Further, one or more segments or regions of the capture probe can optionally be released from the array feature by cleavage of the cleavage domain. As an example, spatial barcodes and/or universal molecular identifiers (UMIs) can be released by cleavage of the cleavage domain.

D. Padlock Oligonucleotides

In some embodiments, the methods provided herein include hybridizing a padlock oligonucleotide to the target analyte bound to the capture domain such that the padlock oligonucleotide is circularized, wherein the padlock oligonucleotide comprises: (i) a first sequence that is substantially complementary to a first portion of the analyte, or a complement thereof, (ii) a backbone sequence, and (iii) a second sequence that is substantially complementary to a second portion of the analyte, or a complement thereof.

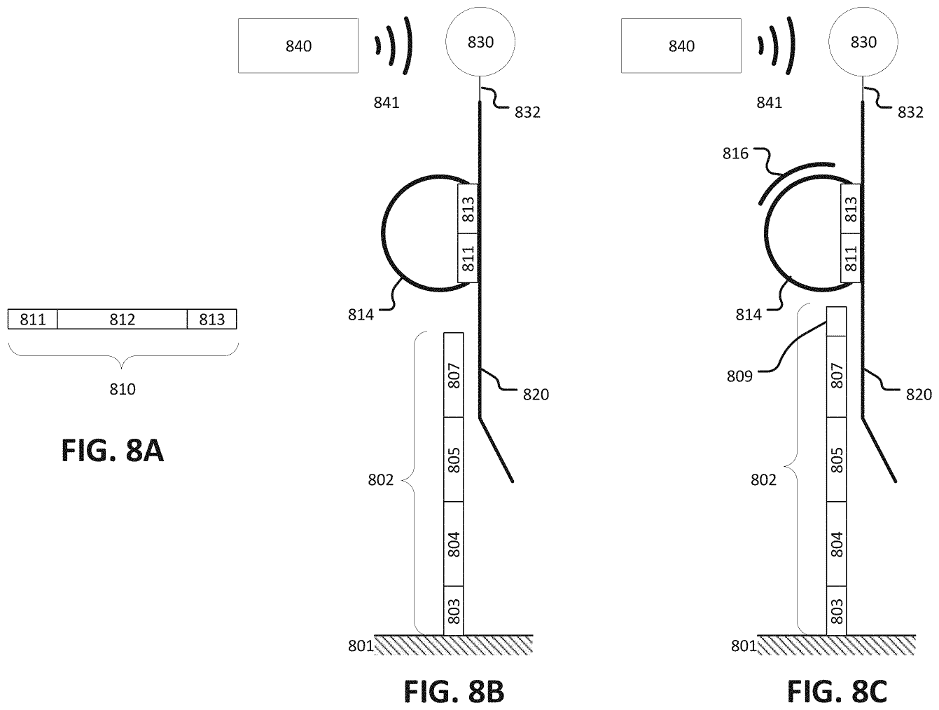

In some embodiments, following hybridizing the target analyte 720 to the capture domain 707 on the capture probe 702 and applying the stretching force 742 to the stretching moiety 730 thereby eliminating secondary structure 722 present, a padlock oligonucleotide is introduced to the solution. As shown in FIGS. 8A and 8B, a padlock oligonucleotide 810 includes a first sequence 811 that is substantially complementary to a first portion of the target analyte 820, a backbone sequence 812, and a second sequence 813 that is substantially complementary to a second portion of the target analyte 820, the first portion and the second portion being adjacent. Therefore, the first sequence and the second sequence are directly adjacent when hybridized to the target analyte. As a result, the second sequence 813 can be ligated to the first sequence 811, thereby creating a circularized padlock oligonucleotide 814.

As shown in FIG. 8C, following circularization of the padlock oligonucleotide, an amplification primer 816 is hybridized to the circularized padlock oligonucleotide 814. Rolling circle amplification (RCA) is used to amplify the circularized padlock oligonucleotide 814 using, for example, a Phi29 DNA polymerase. To prevent the capture probe 802 from being extended during the amplification step, the capture probe includes a blocking moiety 809 on the 3′ end. RCA synthesizes continuous single-stranded copies (e.g., amplified circularized padlock oligonucleotide) of the circularized padlock oligonucleotide 814. Following RCA, the amplified circularized padlock oligonucleotide is contacted with a plurality of detection probes, where a detection probe of the plurality of detection probes include a sequence that is substantially complementary to a sequence of the padlock oligonucleotide and a fluorophore.

As used herein, a “padlock oligonucleotide” refers to an oligonucleotide that has, at its 5′ and 3′ ends, sequences (e.g., a first sequence at the 5′ end and a second sequence at the 3′ end) that are complementary to adjacent or nearby portions (e.g., a first portion and a second portion) of the analyte or analyte derived molecule. Upon hybridization to the first and second portions of the analyte or analyte derived molecule, the two ends of the padlock oligonucleotide are either brought into contact or an end is extended until the two ends are brought into contact, allowing circularization of the padlock oligonucleotide by ligation (e.g., ligation using any of the methods described herein). The ligation product can be referred to as the “circularized padlock oligonucleotide.” In some embodiments, after circularization of the oligonucleotide, rolling circle amplification is used to amplify the circularized padlock oligonucleotide.

In some embodiments, a first sequence of a padlock oligonucleotide includes a sequence that is substantially complementary to a first portion of the analyte or analyte derived molecule. In some embodiments, the first portion of the analyte or analyte derived molecule is 5′ to the second portion of the analyte or the analyte derived molecule. In some embodiments, the first sequence is at least 70% identical (e.g., at least 75% identical, at least 80% identical, at least 85% identical, at least 90% identical, at least 95% identical, or at least 99% identical) to the first portion.

In some embodiments, a backbone sequence of a padlock oligonucleotide includes a sequence that is substantially complementary to an amplification primer. The amplification primer can be a primer used in a rolling circle amplification reaction (RCA), where the RCA increases the “copy number” of the analyte or analyte derived molecule. In some embodiments, the backbone sequence includes a functional sequence.

In some embodiments, a second sequence of a padlock oligonucleotide includes a sequence that is substantially complementary to a second portion of the analyte or analyte derived molecule. In some embodiments, the second portion of the analyte or analyte derived molecule is 3′ to the first portion of the analyte or the analyte derived molecule. In some embodiments, the second sequence is at least 70% identical (e.g., at least 75% identical, at least 80% identical, at least 85% identical, at least 90% identical, at least 95% identical, or at least 99% identical) to the second portion.

In some embodiments, the first sequence is substantially complementary to a first portion of the analyte or analyte derived molecule that is directly adjacent to the second portion of the analyte or analyte derived molecule to which the second sequence is substantially complementary. In such cases, the first sequence is ligated to the second sequence.

In some embodiments, the first sequence is substantially complementary to a first portion of the analyte or analyte derived molecule that is not directly adjacent to the second portion of the analyte or analyte derived molecule to which the second sequence is substantially complementary. In such cases, a “gap” exists between where the first sequence is hybridized to the first potion and where the second sequence is hybridized to the second portion. In some embodiments, there is a sequence (e.g., a gap) in the analyte between the first portion and the second portion of at least 1-100, 1-90, 1-80, 1-70, 1-60, 1-50, 1-40, 1-30, 1-20, 1-10, 1-9, 1-8, 1-7, 1-6, 1-5, 1-4, 1-3, 1-2 or 1 nucleotide(s). In a non-limiting example, a first sequence having a sequence that is complementary to a sequence 5′ of the gap and a second sequence having a sequence that is complementary to a sequence 3′ of the gap each bind to an analyte leaving a sequence (e.g., the “gap”) in between the first and second sequences that is gap-filled thereby perrmitting ligation and generation of the circularized padlock oligonucleotide. In some instances, to generate a padlock oligonucleotide that includes a first sequence and a second sequence that are close enough to one another to initiate a ligation step, the second sequence is extended enzymatically (e.g., using a polymerase as known in the art).

In some embodiments, the “gap” sequence between the first sequence and the second sequence include one nucleotide, two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, at least 25 nucleotides, at least 30 nucleotide, at least 35 nucleotides, at least 40 nucleotides, at least 45 nucleotides, or at least 50 nucleotides.

In some embodiments, extending the second sequence of the padlock oligonucleotide includes a nucleic acid extension reaction (e.g., any of the nucleic acid extension reactions described herein). In some embodiments, extending the second sequence of the padlock oligonucleotide includes using a reverse transcriptase (e.g., any of the reverse transcriptase described herein). In some embodiments, extending the second sequence of the padlock oligonucleotide includes using a Moloney Murine Leukemia Virus (M-MulV) reverse transcriptase. In some embodiments, extending the second sequence of the padlock oligonucleotide generates a sequence that is complementary to the analyte or the analyte derived molecule. In some embodiments, extending the second sequence of the padlock oligonucleotide generates an extended second sequence of the padlock oligonucleotide that is complementary to the analyte or analyte derived molecule. In some embodiments, second sequence of the padlock oligonucleotide generates a sequence that is adjacent to the first sequence of the padlock oligonucleotide.

Once the first and second sequences in a padlock oligonucleotide are adjacent, ligation of the two ends can occur. In some embodiments, the ligation step includes ligating the second sequence to the first sequence of the padlock oligonucleotide using enzymatic or chemical ligation. In some embodiments where the ligation is enzymatic, the ligase is selected from a T4 RNA ligase (Rnl2), a PBCV-1 ligase or a Chlorella virus ligase (also called SplintR ligase in some instances), a single stranded DNA ligase, or a T4 DNA ligase. In some embodiments, the ligase is a T4 RNA ligase (Rnl2) ligase. In some embodiments, the ligase is a pre-activated T4 DNA ligase as described herein. A non-limiting example describing methods of generating and using pre-activated T4 DNA include U.S. Pat. No. 8,790,873, the entire contents of which are herein incorporated by reference.

In a non-limiting example, the methods further include an amplifying step where one or more amplification primers are hybridized to the circularized padlock oligonucleotide or RCA product generated thereof and the circularized padlock oligonucleotide or RCA product generated thereof is amplified using a polymerase. The amplifying step increases the copy number of the analyte or analyte derived molecule for detection. The increased copy number can be detected by detection probes and used to identify the location of the analyte in the biological sample and thereby determine whether the methods for releasing analytes from a biological sample has been successful. In some embodiments, the amplifying step is rolling circle amplification (RCA). In some embodiments, the amplifying step is strand displacement amplification, or multiple displacement amplification.

As used herein, rolling circle amplification (RCA) refers to a polymerization reaction carried out using a single-stranded circular DNA (e.g., a circularized padlock oligonucleotide) as a template and an amplification primer that is substantially complementary to the single-stranded circular DNA (e.g., the circularized padlock oligonucleotide) to synthesize multiple concatenated single-stranded copies of the template DNA (e.g., the circularized padlock oligonucleotide). In some embodiments, RCA includes hybridizing one or more amplification primers to the circularized padlock oligonucleotide and amplifying the circularized padlock oligonucleotide using a polymerase with strand displacement activity, such as Phi29 DNA polymerase, Bst DNA polymerases (e.g., large fragment, 2.0 and 3.0), Klenow fragment, and Vent or DeepVent DNA polymerases.

In some embodiments, an amplification primer includes a sequence that is substantially complementary to one or more of the first sequence, the backbone sequence, or the second sequence of the padlock oligonucleotide. For example, the amplification primer can be substantially complementary to the backbone sequence. In some embodiments, the amplification primer includes a sequence that is substantially complementary to the padlock oligonucleotide and an additional portion of the analyte or analyte derived molecule. For example, the amplification primer can be substantially complementary to the backbone sequence and a portion of the analyte or analyte derived molecule that does not include the first and second portion. In some embodiments, the amplification primer includes a sequence that is substantially complementary to two or more of the first sequence, the backbone sequence, or the second sequence of the padlock oligonucleotide and an additional portion of the analyte or analyte derived molecule. By substantially complementary, it is meant that the amplification primer is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a sequence in the circularized padlock oligonucleotide.

This disclosure additionally include methods for determining a presence or abundance of an analyte in a biological sample by detection of a signal that corresponds to an amplified circularized padlock oligonucleotide (which thereby corresponds to an analyte or analyte derived molecule). In some embodiments, the method includes a step of detecting a signal corresponding to the amplified circularized padlock oligonucleotide on the substrate, thereby identifying whether a reaction condition, such as a permeabilization condition, results in the detection of an analyte in the biological sample. In some instances, the signal is a detectable signal (e.g., a fluorescent signal) using a detectable label described herein.

In some embodiments, the detecting step includes contacting the amplified circularized padlock oligonucleotide with a plurality of detection probes in order to quantify (or quantitate) the signal. In some instances, quantitating the signal can be performed on an absolute scale. For instances, a total number of fluorescent pixels can be calculated to provide a readout/quantity of the location and abundance of an analyte in a biological sample. In some instances, the quantitating can be performed on a relative scale using a positive or negative control sample. Any method of signal detection can be used herein.

In some embodiments, a detection probe of the plurality of detection probes includes a sequence that is substantially complementary to a sequence of the padlock oligonucleotide, circularized padlock oligonucleotide, or amplified circularized padlock oligonucleotide and a detectable label. For example, the detection probe of the plurality of detection probes can include a sequence that is substantially complementary to a sequence of an amplified circularized padlock oligonucleotide and a detectable label. By substantially complementary, it is meant that the detection probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a sequence in the padlock oligonucleotide, circularized padlock oligonucleotide or the amplified circularized padlock oligonucleotide.

In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF® -97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red) , Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

In some embodiments, the detectable label includes a luminescent or chemiluminescent moiety. Common luminescent/chemiluminescent moieties include, but are not limited to, peroxidases such as horseradish peroxidase (HRP), soybean peroxidase (SP), alkaline phosphatase, and one or more luciferases. These protein moieties can catalyze chemiluminescent reactions given the appropriate chemical substrates (e.g., an oxidizing reagent plus a chemiluminescent or bioluminescent compound). A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, lucigenins, or acridinium esters.

In some embodiments, the plurality of detection probes include a pool of detection probes where each detection probe includes a sequence different from the other detection probes, thereby enabling detection of signals from two or more different sequences (e.g., two or more different amplified circularized padlock oligonucleotides). In some embodiments, each of the two or more different sequences is located on the same amplified circularized padlock oligonucleotide. In some embodiments, each of the two or more different sequences is located on two or more different amplified circularized padlock oligonucleotide (e.g., each amplified circularized padlock oligonucleotide is derived from a different analyte molecule).

In some embodiments, the detecting step includes contacting the amplified circularized padlock oligonucleotide with a detectable label that can label nucleic acid sequences in a non-sequence dependent manner. In such cases, the single-stranded copies of the template DNA produced by RCA can be detected using fluorescent dyes that non-specifically bind to the single-stranded nucleic acid (e.g., the amplified circularized padlock oligonucleotide). For example, in the case where an analyte is an amplified circularized padlock oligonucleotide, a fluorescent dye can bind, either directly or indirectly, to the single stranded nucleic acid. The dye can then be detected as a signal corresponding to the amplified circularized padlock oligonucleotide (i.e., the location and/or the abundance of the circularized padlock oligonucleotide). Non-limiting examples of fluorescent dyes that can bind to single stranded nucleic acids include: TOTO®-1/TO-PRO®-1, TOTO®-3/TO-PRO®-3, TO-PRO®-5, Ethidium bromide, Ethidium homodimer-1 (EthD-1), YOYO®-1/YO-PRO®-1, YOYO®-3/YO-PRO®-3, 7-AAD (7-Aminoactinomycin D), and OliGreen®.

In some embodiments, the detecting step includes contacting the amplified circularized padlock oligonucleotide with a detection probe (e.g., any of the exemplary detection probes described herein) and a fluorescent dye that can label single-stranded DNA in an non-sequence dependent manner (e.g., any of the exemplary fluorescent dyes described herein).

In some embodiments, detecting the signal or signals that corresponding to the amplified circularized padlock oligonucleotide on the substrate includes obtaining an image corresponding to the analyte and/or analyte derived molecule on the first substrate. In some embodiments, the method further includes registering image coordinates to a fiducial marker.

In some embodiments, the method includes repeating the detecting step with a second plurality of detection probes. In such cases, the method includes removing the detection probes from the first detecting step and contacting the amplified circularized padlock oligonucleotide with a second plurality of detection probes. A detection probe of the second plurality of detection probes can include a sequence that is substantially complementary to a sequence of the padlock oligonucleotide that is non-overlapping, partially overlapping, or completely overlapping with the sequence to which a detection probe from the first plurality of detection probes is substantially complementary.

E. Biological Samples

Methods disclosed herein can be performed on any type of sample. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample. In some embodiments, where the tissue sample is the FFPE tissue sample, and the tissue sample is decrosslinked.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. In some instances, the biological sample can include one or more diseased cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogeneous sample. In some instances, the biological sample is a heterogeneous sample that includes tumor or cancer cells and/or stromal cells.

In some instances, the cancer is breast cancer. In some instances, the breast cancer is triple positive breast cancer (TPBC). In some instances, the breast cancer is triple negative breast cancer (TNBC).

In some instances, the cancer is colorectal cancer. In some instances, the cancer is ovarian cancer. In certain embodiments, the cancer is squamous cell cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, bladder cancer, breast cancer, colon cancer, colorectal cancer, endometrial carcinoma, myeloma, salivary gland carcinoma, kidney cancer, basal cell carcinoma, melanoma, prostate cancer, vulval cancer, thyroid cancer, testicular cancer, esophageal cancer, or a type of head or neck cancer. In certain embodiments, the cancer treated is desmoplastic melanoma, inflammatory breast cancer, thymoma, rectal cancer, anal cancer, or surgically treatable or non-surgically treatable brain stem glioma. In some embodiments, the subject is a human.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E). The methods disclosed herein are compatible with H&E which will allow for morphological context to be overlaid with transcriptomic analysis. However, depending on the need some samples may be stained with only a nuclear stain, such as staining a sample with only hematoxylin and not eosin, when location of a cell nucleus is needed.

In some embodiments, a biological sample (e.g. tissue section) can be fixed with methanol, stained with hematoxylin and eosin, and imaged. In some embodiments, fixing, staining, and imaging occurs before one or more probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the FFPE sample is deparaffinized, permeabilized, equilibrated, and blocked before target probe oligonucleotides are added. In some embodiments, deparaffinization using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, permeabilizing a sample includes washing the sample with a phosphate buffer. In some embodiments, the buffer is PBS. In some embodiments, the buffer is PBST.

In some embodiments, the biological sample was previously stained. In some embodiments, the biological sample was previously stained using immunofluorescence or immunohistochemistry. In some embodiments, the biological sample was previously stained using hematoxylin and eosin.

F. Permeabilization

In some embodiments, the method also includes contacting the biological sample with a permeabilization agent, wherein the permeabilization agent is selected from an organic solvent, a detergent, and an enzyme, or a combination thereof. Non-limiting examples of permeabilization agents include without limitation: an endopeptidase, a protease sodium dodecyl sulfate (SDS), polyethylene glycol tert-octylphenyl ether, polysorbate 80, and polysorbate 20, N-lauroylsarcosine sodium salt solution, saponin, Triton X-100™, and Tween-20™. In some embodiments, the endopeptidase is pepsin or proteinase K. In some embodiments, the permeabilizing step is performed after contacting the biological sample with the substrate.

G. Determining the Sequence and/or an Amount of an Analyte

Some embodiments of the methods described herein further include determining an amount of the extended capture probe released from the substrate. In some embodiments, the amount of extended capture probe released from the substrate can be determined using, e.g., nucleic acid amplification. In some embodiments, the amount of extended capture probe released from the substrate can be determined using optical methods, e.g., hybridization of a fluorophore-conjugated probe.

Some embodiments of any of the methods described herein can further include comparing the amount of extended capture probe released from the substrate to a reference level. The reference level can be, e.g., produced by a control method that can include the performance of the steps described herein but can use one or more different parameter or one or more different steps. For example, the different parameter can include a different biological sample, a different set of reagents, a different condition, or any combination thereof.

In some embodiments, the step of determining comprises sequencing (i) all or a part of the sequence corresponding to the target analyte specifically bound by the capture domain or the complement thereof, and (ii) all or a part of the sequence corresponding to the spatial barcode or the complement thereof. In some embodiments, the step of determining comprises sequencing (i) all or a part of the sequence corresponding to the analyte binding moiety barcode or the complement thereof, and (ii) all or part of the sequence corresponding to the spatial barcode or the complement thereof. In some embodiments, the sequencing is high throughput next generation sequencing (e.g., sequence by synthesis, sequence by hybridization, sequence by ligation, nanopore sequencing, single molecule sequencing, etc.).

After an analyte from the biological sample has hybridized or otherwise been associated with a capture probe according to any of the methods described above in connection with the general spatial cell-based analytical methodology, the barcoded constructs that result from hybridization/association are analyzed. In some embodiments, the barcoded constructs include an analyte from the first region of interest. In some embodiments, the barcoded constructs include an analyte from the first region of interest, an analyte from the second region, or combinations thereof.

In some embodiments, the methods provided herein include determining, from one or more first regions of interest, (i) all or a portion of a sequence corresponding to the analyte bound to the capture domain or a complement thereof, and (ii) all or a portion of a sequence corresponding to the spatial barcode or a complement thereof, and using (e.g., correlating) the determined sequences of (i) and (ii) to determine the abundance or location of the analyte in the first region of interest in the biological sample. As such, the sequences of the spatial barcode and the sequence corresponding to the analyte are used to correlate the location of the analyte with its location in the tissue that was proximal and superior to the location of the spatial barcode on the array.

In some embodiments, provided herein are methods for spatially detecting an analyte (e.g., detecting the location of an analyte, e.g., a biological analyte) from a biological sample (e.g., present in a biological sample), the method comprising: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality of capture probes captures the biological analyte; and (d) analyzing the captured biological analyte, thereby spatially detecting the biological analyte; wherein the biological sample is fully or partially removed from the substrate.

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, provided herein are methods for spatially detecting an analyte (e.g., detecting the location of an analyte, e.g., a biological analyte) from a biological sample (e.g., present in a biological sample) that include: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality of capture probes captures the biological analyte; and (d) analyzing the captured biological analyte, thereby spatially detecting the biological analyte; where the biological sample is not removed from the substrate.

In some embodiments, provided herein are methods for spatially detecting a biological analyte of interest from a biological sample that include: (a) staining and imaging a biological sample on a substrate; (b) providing a solution comprising a permeabilization reagent to the biological sample on the substrate; (c) contacting the biological sample with an array on a substrate, wherein the array comprises one or more capture probe pluralities thereby allowing the one or more pluralities of capture probes to capture the biological analyte of interest; and (d) analyzing the captured biological analyte, thereby spatially detecting the biological analyte of interest; where the biological sample is not removed from the substrate.

In some embodiments, the method further includes subjecting a region of interest in the biological sample to spatial transcriptomic analysis. In some embodiments, one or more of the capture probes includes a capture domain. In some embodiments, one or more of the capture probes comprises a unique molecular identifier (UMI). In some embodiments, one or more of the capture probes comprises a cleavage domain. In some embodiments, the cleavage domain comprises a sequence recognized and cleaved by a uracil-DNA glycosylase, apurinic/apyrimidinic (AP) endonuclease (APE1), U uracil-specific excision reagent (USER), and/or an endonuclease VIII. In some embodiments, one or more capture probes do not comprise a cleavage domain and is not cleaved from the array.

In some embodiments, a capture probe can be extended (an “extended capture probe,” e.g., as described herein). For example, extending a capture probe can include generating cDNA from a captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending a capture probe, e.g., the cDNA generation, the captured (hybridized) nucleic acid, e.g., RNA, acts as a template for the extension, e.g., reverse transcription, step.

In some embodiments, the capture probe is extended using reverse transcription. For example, reverse transcription includes synthesizing cDNA (complementary or copy DNA) from RNA, e.g., (messenger RNA), using a reverse transcriptase. In some embodiments, reverse transcription is performed while the tissue is still in place, generating an analyte library, where the analyte library includes the spatial barcodes from the adjacent capture probes. In some embodiments, the capture probe is extended using one or more DNA polymerases.

In some embodiments, a capture domain of a capture probe includes a primer for producing the complementary strand of a nucleic acid hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

In some embodiments, a full-length DNA (e.g., cDNA) molecule is generated. In some embodiments, a “full-length” DNA molecule refers to the whole of the captured nucleic acid molecule. However, if a nucleic acid (e.g., RNA) was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, Wis.). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9oN) DNA ligase (9oN™ DNA ligase, New England Biolabs), Ampligase™ (available from Lucigen, Middleton, Wis.), and PBCV-1 ligase or a Chlorella virus ligase (e.g., SplintR (available from New England Biolabs, Ipswich, Mass.)). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments, double-stranded extended capture probes are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe (e.g., RNA-cDNA hybrid) using a primer including the affinity group. In some embodiments, the primer includes an affinity group and the extended capture probes includes the affinity group. The affinity group can correspond to any of the affinity groups described previously.

In some embodiments, the extended capture probes including the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended capture probes can function to release the extended probes from the surface of the substrate, insofar as copies of the extended probes are not immobilized on the substrate.

In some embodiments, the extended capture probe or complement or amplicon thereof is released. The step of releasing the extended capture probe or complement or amplicon thereof from the surface of the substrate can be achieved in a number of ways. In some embodiments, an extended capture probe or a complement thereof is released from the array by nucleic acid cleavage and/or by denaturation (e.g., by heating to denature a double-stranded molecule).

In some embodiments, the extended capture probe or complement or amplicon thereof is released from the surface of the substrate (e.g., array) by physical means. For example, where the extended capture probe is indirectly immobilized on the array substrate, e.g., via hybridization to a surface probe, it can be sufficient to disrupt the interaction between the extended capture probe and the surface probe. Methods for disrupting the interaction between nucleic acid molecules include denaturing double stranded nucleic acid molecules are known in the art. A straightforward method for releasing the DNA molecules (i.e., of stripping the array of extended probes) is to use a solution that interferes with the hydrogen bonds of the double stranded molecules. In some embodiments, the extended capture probe is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended capture probe from the substrate.

In some embodiments, where the extended capture probe includes a cleavage domain, the extended capture probe is released from the surface of the substrate by cleavage. For example, the cleavage domain of the extended capture probe can be cleaved by any of the methods described herein. In some embodiments, the extended capture probe is released from the surface of the substrate, e.g., via cleavage of a cleavage domain in the extended capture probe, prior to the step of amplifying the extended capture probe.

In some embodiments, probes complementary to the extended capture probe can be contacted with the substrate. In some embodiments, the biological sample can be in contact with the substrate when the probes are contacted with the substrate. In some embodiments, the biological sample can be removed from the substrate prior to contacting the substrate with probes. In some embodiments, the probes can be labeled with a detectable label (e.g., any of the detectable labels described herein). In some embodiments, probes that do not specially bind (e.g., hybridize) to an extended capture probe can be washed away. In some embodiments, probes complementary to the extended capture probe can be detected on the substrate (e.g., imaging, any of the detection methods described herein).

In some embodiments, probes complementary to an extended capture probe can be about 4 nucleotides to about 100 nucleotides long. In some embodiments, probes (e.g., detectable probes) complementary to an extended capture probe can be about 10 nucleotides to about 90 nucleotides long. In some embodiments, probes (e.g., detectable probes) complementary to an extended capture probe can be about 20 nucleotides to about 80 nucleotides long. In some embodiments, probes (e.g., detectable probes) complementary to an extended capture probe can be about 30 nucleotides to about 60 nucleotides long. In some embodiments, probes (e.g., detectable probes) complementary to an extended capture probe can be about 40 nucleotides to about 50 nucleotides long. In some embodiments, probes (e.g., detectable probes) complementary to an extended capture probe can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 nucleotides long.

In some embodiments, about 1 to about 100 probes can be contacted to the substrate and specifically bind (e.g., hybridize) to an extended capture probe. In some embodiments, about 1 to about 10 probes can be contacted to the substrate and specifically bind (e.g., hybridize) to an extended capture probe. In some embodiments, about 10 to about 100 probes can be contacted to the substrate and specifically bind (e.g., hybridize) to an extended capture probe. In some embodiments, about 20 to about 90 probes can be contacted to the substrate and specifically bind (e.g., hybridize) to an extended capture probe. In some embodiments, about 30 to about 80 probes (e.g., detectable probes) can be contacted to the substrate and specifically bind (e.g., hybridize) to an extended capture probe. In some embodiments, about 40 to about 70 probes can be contacted to the substrate and specifically bind (e.g., hybridize) to an extended capture probe. In some embodiments, about 50 to about 60 probes can be contacted to the substrate and specifically bind (e.g., hybridize) to an extended capture probe. In some embodiments, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 probes can be contacted to the substrate and specifically bind (e.g., hybridize) to an extended capture probe.

In some embodiments, the probes can be complementary to a single analyte (e.g., a single gene). In some embodiments, the probes can be complementary to one or more analytes (e.g., analytes in a family of genes). In some embodiments, the probes (e.g., detectable probes) can be for a panel of genes associated with a disease (e.g., cancer, Alzheimer's disease, Parkinson's disease).

In some instances, the ligated probe and capture probe can be amplified or copied, creating a plurality of cDNA molecules. In some embodiments, cDNA can be denatured from the capture probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flow cell (Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

In some embodiments, where a sample is barcoded directly via hybridization with capture probes or analyte capture agents hybridized, bound, or associated with either the cell surface, or introduced into the cell, as described above, sequencing can be performed on the intact sample.

A wide variety of different sequencing methods can be used to analyze barcoded analyte (e.g., the ligation product). In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, Nanopore sequencing, and Polony sequencing), ligation methods, and microarray methods.

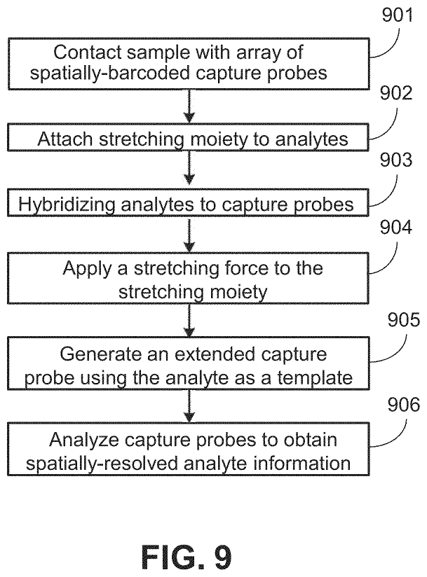

FIG. 9 shows an exemplary workflow that utilizes a spatially-barcoded array on a substrate. The spatially-barcoded capture probes can include a cleavage domain, one or more functional domains, a spatial barcode, a unique molecular identifier, and a capture domain. The spatially-barcoded capture probes can also include a 5′ end modification for reversible attachment to the substrate. The spatially-barcoded array is contacted with a biological sample 901, and the sample is permeabilized through application of permeabilization reagents. Permeabilization reagents may be administered by placing the array/sample assembly within a bulk solution. Alternatively, permeabilization reagents may be administered to the sample via a diffusion-resistant medium and/or a physical barrier such as a lid, wherein the sample is sandwiched between the diffusion-resistant medium and/or barrier and the array-containing substrate.

A stretching moiety 730 (as shown in FIGS. 7A-7F), is attached to a plurality of target analytes 902 released from the biological sample during permeabilization. The stretching moiety-bound target analytes are migrated toward the spatially-barcoded capture array using any number of techniques disclosed herein. For example, analyte migration can occur using a diffusion-resistant medium lid and passive migration (e.g., gravitational forces). As another example, analyte migration can be active migration, using an electrophoretic transfer system, for example. Once the analytes are in close proximity to the spatially-barcoded capture probes, the capture probes can hybridize or otherwise bind a target analyte 903. The biological sample can be optionally removed from the array.

In some embodiments, steps 902 and 903 can happen simultaneously, or in a step-wise manner. For example, analyte migration can occur concurrently with, or after, biological sample permeabilization. In some embodiments, the analyte migration can occur concurrently with, or after, to binding the stretching moiety to the target analyte. In embodiments in which the stretching moiety binds specifically to a linkage site within or attached to the target analyte, the stretching moiety is attached during or before the active migration.

Alternatively, in some embodiments, the solution includes a blocking probe which blocks or modifies the free 3′ end of the capture domain of the capture probe preventing binding the target analyte. The stretching moiety is introduced to the solution before or after removal of the biological sample from the array which binds to the target analytes present. The blocking probe is then removed from the capture probe which facilitates binding of the target analyte including the stretching moiety to the capture domain.

A stretching force, such as stretching force 742 (as shown in FIG. 7D), is applied to the stretching moiety 904, thereby elongating the analyte hybridized to the capture domain. Referring for instance to FIG. 7D, a field application instrument 740 generates an applied field 741 which creates a stretching force on the stretching moiety. The stretching force has a magnitude and a direction, as described herein. The stretching force 742 creates a tension in the target analyte which eliminates secondary structure, such as secondary structure 722, present in the target analyte. The stretching force elongates, e.g., stretches, the target analyte 720 to at least a portion of the target analyte maximum length.

The capture probes can be optionally cleaved from the array, and the captured analytes can be spatially-barcoded by performing a reverse transcriptase first strand cDNA reaction. A first strand cDNA reaction can be optionally performed using template switching oligonucleotides. For example, a template switching oligonucleotide can hybridize to a poly(C) tail added to a 3′ end of the cDNA by a reverse transcriptase enzyme in a template independent manner. The original mRNA template and template switching oligonucleotide can then be denatured from the cDNA and the spatially-barcoded capture probe can then hybridize with the cDNA and a complement of the cDNA can be generated. The first strand cDNA can then be purified and collected for downstream amplification steps. The first strand cDNA can be amplified using PCR 906, where the forward and reverse primers flank the spatial barcode and analyte regions of interest, generating a library associated with a particular spatial barcode. In some embodiments, the library preparation can be quantitated and/or quality controlled to verify the success of the library preparation steps. In some embodiments, the cDNA comprises a sequencing by synthesis (SBS) primer sequence. The library amplicons are sequenced and analyzed to decode spatial information.

Alternatively, the captured analytes are used as in a second strand cDNA synthesis reaction while the capture probe is affixed to the array. The capture probe is extended through reverse transcription using the captured analyte as a template. A poly(C) tail can be added to a 3′ end of the capture probe by a reverse transcriptase enzyme. A template switching oligonucleotide (TSO) can hybridize to the poly(C) tail in a template independent manner. The capture probe is extended to complement the TSO. The captured analytes including the TSO can be denatured from the capture probe and a second strand primer sequence hybridized to the complementary TSO sequence. The second strand primer sequence is used by a DNA polymerase enzyme to generate a complement of the extended capture probe in a second strand synthesis reaction. The generated second strand cDNA is then denatured from the capture probe and can be amplified for library construction, quantitation, and/or quality control as described herein.

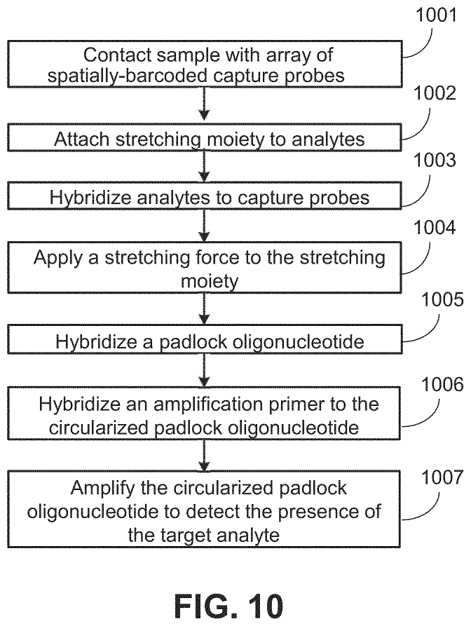

FIG. 10 shows an exemplary workflow that utilizes a stretching moiety and a padlock oligonucleotide to detect the presence of a target analyte. A spatially-barcoded array is contacted with a biological sample 1001, and the sample is permeabilized through application of permeabilization reagents, as described above. A stretching moiety, such as stretching moiety 830, is attached to a plurality of target analytes 1002 released from the biological sample during permeabilization. The stretching moiety-bound target analytes are migrated toward the spatially-barcoded capture array using any number of techniques disclosed herein. Once the analytes are in close proximity to the spatially-barcoded capture probes, the capture probes can hybridize or otherwise bind a target analyte 1003. The biological sample can be optionally removed from the array.

For example, referring to FIG. 8C, a stretching force 842, is applied to the stretching moiety 830 thereby elongating the analyte hybridized to the capture domain and eliminating secondary structures in the target analyte, as described above. The stretching force stretches the target analyte 820 to at least a portion of the target analyte maximum length. A padlock oligonucleotide, including a first sequence and a second sequence, is attached to the target analyte 820. In some embodiments, the first sequence and a second sequence are complementary to a first and a second sequence, respectively, within the target analyte. In some embodiments, the first sequence and a second sequence are complementary to adjacent first and a second sequences. Attaching the padlock oligonucleotide to the target analyte creates a circularized padlock oligonucleotide.

An amplification primer, such as amplification primer 816, is hybridized to the circularized padlock oligonucleotide 814. The amplification primer can include a sequence that is substantially complementary to one or more of the first sequence, the backbone sequence, or the second sequence of the padlock oligonucleotide. For example, the amplification primer can be substantially complementary to the backbone sequence. The amplification primer is used to amplify the circularized padlock oligonucleotide to detect the presence of the target analyte 820. The presence of the target analyte can be detected by contacting the amplified circularized padlock oligonucleotide with a plurality of detection probes using any detection probes as described herein.

H. Kits

Also provided herein are kits that can be used to perform any of the methods described herein. In some embodiments, a kit includes: (a) a plurality of stretching moieties; (b) a plurality of primers; (c) one or more enzymes selected from a polymerase, a reverse transcriptase, and a ligase; (d) a substrate comprising a plurality of capture probes, wherein a capture probe of the plurality of capture probes comprises a capture domain and a spatial barcode; (e) magnetic tweezers; and (f) instructions for performing any of the methods described herein.

### EXAMPLES

### Example 1

### Resolving Secondary Structures of mRNA Molecules by Attaching a Stretching Moiety

This example provides an exemplary method of enhancing spatial detection of target mRNA molecules by resolving the secondary structure of the mRNA molecules. In particular, the secondary structure of the mRNA molecule is resolved by attaching a stretching moiety to the mRNA molecule.

In a non-limiting example, a biological sample (e.g., an FFPE sample) is contacted with a substrate (e.g., a spatial array) including a plurality of capture probes, where each capture probe includes a capture domain and a spatial barcode. The biological sample is then permeabilized and the mRNA molecule is released from the biological sample. In particular, after 30 minutes, the tissues are washed and permeabilized by adding 1.25 mg/ml Proteinase K, incubated at 37° C. for at least 5 minutes and then are washed to remove the protease. The released mRNA molecules are allowed to hybridize to the capture domain on the capture probe immobilized on the spatial array. Excess mRNA molecules not bound to a capture probe are washed off.

Next, a stretching moiety is affixed to the mRNA molecule, as described, e.g., in FIGS. 7A-7E and in Chang et al. (“Single-molecule Mechanical Analysis of Strand Invasion in Human Telomere DNA”. BIORXIV preprint (Jun. 23, 2021), doi.org/10.1101/2021.06.22.449520). FIG. 7A depicts a capture probe 702 comprising a cleavage domain 703, functional sequence 704, spatial barcode 705, and a capture domain 707 immobilized on a substrate 701. The capture domain 707 includes a sequence that specifically hybridizes to the target mRNA molecule 720.

As shown in FIG. 7A, the target mRNA molecule 720 includes a secondary structure 722. The secondary structure 722 occludes a portion of the total length of the mRNA molecule 720, such as occluding a portion of the mRNA molecule 720 from binding to the capture domain 707, or binding to a reverse transcriptase. Also shown in FIG. 7A is a stretching moiety 730. The stretching moiety 730 affixes to one end of the mRNA molecule 720 through a linkage site 732. The linkage site 732 provides a permanent or temporary (e.g., reversible) connection between the stretching moiety 730 and the mRNA molecule 720. FIG. 7A shows the linkage site 732 attached to the stretching moiety 730. However, in additional or alternative embodiments, the linkage site 732 can be attached to the mRNA molecule 720.

As shown in FIG. 7B, the mRNA molecule 720 includes a cap at one end that includes a first half of a binding pair, while the linkage site 732 affixed to the stretching moiety 730 includes a second half of the binding pair, which specifically binds to the first half. The present example can be interpreted to cover the non-limiting examples of binding pairs described in the foregoing sections, including, but not limited to, biotin-streptavidin pair, antibody-target pair (e.g., digoxigenin-anti-digoxigenin), and protein-ligand pair (e.g., biotin-avidin, or biotin-streptavidin). For example, the linkage site 732 can include a biotin moiety and the target analyte 720 can include an avidin moiety, or vice versa.

As shown in FIG. 7B, the stretching moiety 730 is affixed to the target analyte 720 via linkage site 732. Next, as shown in FIG. 7C, the mRNA molecule 720—including the affixed stretching moiety 730—is hybridized to the capture domain 707 of the capture probe 702. The stretching moiety 730 is a moiety responsive to an applied field. In this example, the stretching moiety 730 is composed of a magnetic material (e.g., a magnetic moiety, magnetic bead) and is responsive to a magnetic field. However, in additional or alternative embodiments, the methods disclosed herein can use stretching moiety 730 that is a polystyrene moiety responsive to a directed light field (e.g., optical tweezers), and/or stretching moiety 730 composed of a magnetic material (e.g., neodymium), a paramagnetic material (e.g., aluminum, gold, copper), or a non-magnetic material (e.g., a polymer material) coated in or impregnated with a magnetic or paramagnetic material.

Next, a stretching force is applied to the stretching moiety, thereby elongating the mRNA molecule hybridized to the capture domain. As shown in FIG. 7D, a field application instrument 740 (e.g., a magnetic instrument; e.g., a set of magnetic tweezers) is used to apply a field to the environment of the stretching moiety 730, target analyte 720, and capture probe 702. In this example, the field application instrument 740 is a magnetic instrument (e.g., a set of magnetic tweezers) that applies a magnetic field to the environment of the stretching moiety 730, target analyte 720, and capture probe 702. The field application instrument 740 includes control software to vary the strength and direction of the applied magnetic field 741 and thereby the stretching force 742. The applied field 741 creates a stretching force 742 on the stretching moiety 730 in at least one direction, e.g., a linear force orthogonal to a plane of an upper surface of the substrate 701, a rotational force (e.g., clockwise, or anti-clockwise) around a rotational axis orthogonal to the plane of the upper surface of the substrate 701, or both. As shown in FIG. 7E, the stretching force 742 created by the applied field 741 between the capture probe 702 affixed to the substrate 701 and the stretching moiety 730 translates along the backbone of the mRNA molecule 720, thereby eliminating the secondary structure 722 and elongating the mRNA molecule 720. FIG. 7E shows the elongated mRNA molecule 720 without the secondary structure 722 and the field application instrument 740 applying the applied field 741 to the stretching moiety 730, thereby stretching the mRNA molecule 720 to a length orthogonal to the plane of the upper surface of the substrate 701.

Next, an extended capture probe is generated using the mRNA molecule as a template. An exemplary workflow for the creation of an extended capture probe is shown in FIG. 7A through FIG. 7F. As shown in FIG. 7F, the mRNA molecule 720 is hybridized to the capture domain 707 of the capture probe 702 and a reverse transcriptase enzyme can add a sequence 708 to the 3′ end of the capture probe that is complementary to a sequence of the mRNA molecule 720, to generate an extended capture probe 709 using the mRNA molecule 720 as the template. The extended capture probe 709 is released from the substrate 701 and the target analyte 720 released from the extended capture probe 709. Following the release of the extended capture probe 709 from the substrate, the solution containing the capture probe 709 is transferred to a fresh container, neutralized, and used to generate a sequencing library. Next, all or a part of a sequence of the mRNA molecule or a complement thereof, and the spatial barcode or a complement thereof is sequenced for spatial analysis of the mRNA molecule. Additionally, or in the alternative, the hybridized mRNA molecule with the affixed stretching moiety is released from the extended capture probe and second strand DNA is synthesized (not shown) from the extended capture probe. In this scenario, the second strand DNA, which includes a complement of the extended capture probe 709, is released from the extended capture probe, transferred, and a sequencing library is generated. Next, all or a part of a sequence of the mRNA molecule or a complement thereof, and the spatial barcode or a complement thereof is sequenced for spatial analysis of the mRNA molecule.

### Example 2

### Use of Padlock Oligonucleotide in a Method for Resolving Secondary Structures of mRNA Molecules

This example provides an exemplary method for resolving secondary structure of mRNA molecules where a padlock oligonucleotide is hybridized to the mRNA molecule. In particular, a padlock oligonucleotide is hybridized to the mRNA molecule bound to the capture domain, such that the padlock oligonucleotide is circularized.

The present example describes an exemplary embodiment of the method (e.g., method for resolving secondary structure of mRNA molecules) described in Example 1, wherein, following hybridization of the mRNA molecule 720 to the capture domain 707 on the capture probe 702 and application of the stretching force 742 to the stretching moiety 730 thereby eliminating secondary structure 722 present, a padlock oligonucleotide is introduced to the solution. An exemplary workflow for the method is provided in FIGS. 8A-8B.

As shown in FIGS. 8A and 8B, a padlock oligonucleotide 810 includes a first sequence 811 that is substantially complementary to a first portion of the mRNA molecule 820, a backbone sequence 812, and a second sequence 813 that is substantially complementary to a second portion of the mRNA molecule 820, the first portion and the second portion being adjacent. Therefore, the first sequence and the second sequence are directly adjacent when hybridized to the mRNA molecule. As a result, the second sequence 813 is ligated to the first sequence 811, thereby creating a circularized padlock oligonucleotide 814.

As shown in FIG. 8C, following circularization of the padlock oligonucleotide, an amplification primer 816 is hybridized to the circularized padlock oligonucleotide 814. Rolling circle amplification (RCA) is used to amplify the circularized padlock oligonucleotide 814, using, for example, a Phi29 DNA polymerase. To prevent the capture probe 802 from being extended during the amplification step, the capture probe includes a blocking moiety 809 on the 3′ end. RCA synthesizes continuous single-stranded copies (e.g., amplified circularized padlock oligonucleotide) of the circularized padlock oligonucleotide 814. Following RCA, the amplified circularized padlock oligonucleotide is contacted with a plurality of detection probes, where a detection probe of the plurality of detection probes include a sequence that is substantially complementary to a sequence of the padlock oligonucleotide and a fluorophore.

Once the first and second sequences in a padlock oligonucleotide are adjacent, ligation of the two ends occur. The ligation step includes ligating the second sequence to the first sequence of the padlock oligonucleotide using enzymatic or chemical ligation. Non-limiting examples of ligases that can be used for enzymatic ligation include a T4 RNA ligase (Rnl2), a SplintR ligase, a single stranded DNA ligase, or a T4 DNA ligase.

In a non-limiting example, the methods further include an amplifying step where one or more amplification primers are hybridized to the circularized padlock oligonucleotide or RCA product generated thereof and the circularized padlock oligonucleotide or RCA product generated thereof is amplified using a polymerase. The amplifying step increases the copy number of the mRNA molecule for detection. The increased copy number is detected by detection probes and used to identify the location of the mRNA molecule in the biological sample. Amplification steps useful in the present method include, without limitation, rolling circle amplification (RCA), strand displacement amplification, and multiple displacement amplification.

In a non-limiting example, RCA is used for amplification of the circularized padlock oligonucleotide. For amplification of circularized padlock oligonucleotide using RCA, the circularized padlock oligonucleotide is used as a template, and an amplification primer that is substantially complementary to the circularized padlock oligonucleotide is used to synthesize multiple concatenated single-stranded copies of the template DNA (e.g., the circularized padlock oligonucleotide). The amplification primer can include a sequence that is substantially complementary to one or more of the first sequence, the backbone sequence, or the second sequence of the padlock oligonucleotide. Next, one or more amplification primers is hybridized to the circularized padlock oligonucleotide and the circularized padlock oligonucleotide is amplified using a polymerase with strand displacement activity, such as Phi29 DNA polymerase, Bst DNA polymerases, Klenow fragment, and Vent or DeepVent DNA polymerases.

In a non-limiting example, the method further includes detection of a signal corresponding to the amplified circularized padlock oligonucleotide (which thereby corresponds to the mRNA molecule), and using the detection to determine presence or abundance of the mRNA molecule in the biological sample. For example, detection of a signal corresponding to the amplified circularized padlock oligonucleotide on the substrate identifies whether a reaction condition, such as a permeabilization condition, results in the detection of the mRNA molecule in the biological sample. The signal can be any of the detectable signals (e.g., signal using any of the detectable labels) described herein.

In a non-limiting example, the detecting step includes contacting the amplified circularized padlock oligonucleotide with a plurality of detection probes in order to quantify (or quantitate) the signal. Quantitating of the signal is performed on an absolute scale (e.g., a total number of fluorescent pixels is calculated to provide a readout/quantity of the location and abundance of the mRNA molecule in the biological sample) or on a relative scale (e.g., using a positive or negative control sample).

### Embodiments

Embodiment 1. A method for determining a presence or abundance of an analyte in a biological sample, the method comprising:

(a) providing the biological sample on a substrate comprising a plurality of capture probes, wherein a capture probe of the plurality of capture probes comprises a spatial barcode and a capture domain;

(b) releasing the analyte from the biological sample;

(c) affixing a stretching moiety to the analyte;

(d) hybridizing the analyte to the capture domain;

(e) applying a stretching force to the stretching moiety, thereby elongating the analyte hybridized to the capture domain; and

(f) generating an extended capture probe using the analyte as a template.
Embodiment 2. The method of embodiment 1, further comprising determining (i) all or a part of a sequence of the analyte or a complement thereof, and (ii) the spatial barcode or a complement thereof, and using the determined sequences of (i) and (ii) to determine the presence or abundance of the analyte in the biological sample.
Embodiment 3. The method of embodiment 2, wherein the determining step comprises sequencing (i) all or a part of a sequence of the analyte or a complement thereof, and (ii) the spatial barcode or a complement thereof.
Embodiment 4. The method of embodiment 3, wherein the sequencing is high throughput sequencing.
Embodiment 5. A method for determining a presence or abundance of an analyte in a biological sample, the method comprising:

(a) providing the biological sample on a substrate comprising a plurality of capture probes, wherein a capture probe of the plurality of capture probes comprises a spatial barcode and a capture domain;

(b) releasing the analyte from the biological sample;

(c) affixing a stretching moiety to the analyte;

(d) hybridizing the analyte to the capture domain;

(e) applying a stretching force to the stretching moiety, thereby elongating the analyte hybridized to the capture domain;

(f) hybridizing a padlock oligonucleotide to the analyte hybridized to the capture domain, wherein the padlock oligonucleotide comprises:
(i) a first sequence that is substantially complementary to a first portion of the analyte, or a complement thereof,
(ii) a backbone sequence, and
(iii) a second sequence that is substantially complementary to a second portion of the analyte, or a complement thereof;

(g) ligating the first sequence to the second sequence of the padlock oligonucleotide, thereby generating a circularized padlock oligonucleotide;

(h) amplifying the circularized padlock oligonucleotide, thereby creating an amplified circularized padlock oligonucleotide, and

(i) identifying the presence or abundance of the analyte in the biological sample.
Embodiment 6. The method of embodiment 5, wherein the identifying the presence or abundance of the analyte comprises determining (i) all or a part of a sequence of the analyte or a complement thereof, and (ii) the spatial barcode or a complement thereof, and using the determined sequences of (i) and (ii) to determine the presence or abundance of the analyte in the biological sample.
Embodiment 7. The method of embodiment 5, wherein the identifying the presence or abundance of the analyte comprises detecting a signal corresponding to the amplified circularized padlock oligonucleotide on the substrate.
Embodiment 8. The method of any one of embodiments 5-7, wherein the amplifying the circularized padlock oligonucleotide comprises rolling circle amplification.
Embodiment 9. The method of embodiment 7 or 8, further comprising quantitating the signal.
Embodiment 10. The method of any one of embodiments 5-9, wherein the first sequence of the padlock oligonucleotide and the second sequence of the padlock oligonucleotide are substantially complementary to adjacent sequences of the analyte.
Embodiment 11. The method of any one of embodiments 5-9, wherein first sequence of the padlock oligonucleotide and the second sequence of the padlock oligonucleotide are substantially complementary to sequences of the analyte that are not adjacent to one another, generating a gap between the first sequence and the second sequence upon hybridization of the first sequence and the second sequence to the analyte, wherein the gap is filled using a polymerase.
Embodiment 12. The method of any one of embodiments 5-11, wherein the ligating step comprises enzymatic ligation or chemical ligation.
Embodiment 13. The method of embodiment 12, wherein the enzymatic ligation utilizes T4 DNA ligase.
Embodiment 14. The method of any one of the preceding embodiments, wherein the stretching moiety is a magnetic bead, and wherein the stretching force is a magnetic force.
Embodiment 15. The method of any one of the preceding embodiments, wherein the stretching force is a linear force orthogonal to a plane of an upper surface of the substrate, a rotational force around a rotational axis orthogonal to the plane of the upper surface of the substrate, or both.
Embodiment 16. The method of any one of the preceding embodiments, wherein the affixing the stretching moiety to the analyte comprises affixing a first binding moiety to a second binding moiety,

wherein the stretching moiety comprises the first binding moiety, and

wherein the analyte comprises the second binding moiety associated with a 5′ end of the analyte or a 3′ end of the analyte.
Embodiment 17. The method of embodiment 16, wherein the first binding moiety comprises digoxigenin, anti-digoxigenin, biotin, avidin, or streptavidin.
Embodiment 18. The method of embodiment 16 or 17, wherein the second binding moiety comprises digoxigenin, anti-digoxigenin, biotin, avidin, or streptavidin.
Embodiment 19. The method of any one of the preceding embodiments, wherein the stretching moiety further comprises a cleavable linker.
Embodiment 20. The method of any one of the preceding embodiments, wherein the stretching force is in a range from 0.05 piconewtons (pN) to 100 pN.
Embodiment 21. The method of embodiment 20, wherein the range is from 0.1 pN to 0.5 pN.
Embodiment 22. The method of embodiment 20 or 21, wherein the range is from 0.2 pN to 0.4 pN.
Embodiment 23. The method of any one of the preceding embodiments, wherein the stretching force is applied for about 1 second (s) to about 10 minutes (min), from about 30 s to about 5 min, or from about 1 min to about 3 min.
Embodiment 24. The method of any one of the preceding embodiments, wherein the stretching force is applied using one of a magnetic field, an electric field, or a light field.
Embodiment 25. The method of embodiment 24, wherein the stretching force is a modulated stretching force.
Embodiment 26. The method of any one of embodiments 1-4 or 14-25, further comprising releasing the extended capture probe from the substrate.
Embodiment 27. The method of any one of the preceding embodiments, wherein the releasing the analyte from the biological sample comprises treating the biological sample with a solution comprising pepsin or proteinase K.
Embodiment 28. The method of any one of the preceding embodiments, wherein the capture domain comprises a poly(A) sequence.
Embodiment 29. The method of any one of the preceding embodiments, wherein the analyte is an RNA.
Embodiment 30. The method of embodiment 29, wherein the RNA is mRNA.
Embodiment 31. The method of any one of embodiments 1-28, wherein the analyte is DNA.
Embodiment 32. The method of embodiment 31, wherein the DNA is genomic DNA.
Embodiment 33. The method of any one of the preceding embodiments, wherein the biological sample is a tissue sample.
Embodiment 34. The method of embodiment 33, wherein the tissue sample is a fixed tissue sample.
Embodiment 35. The method of embodiment 34, wherein the fixed tissue sample is a formalin-fixed paraffin-embedded (FFPE) sample.
Embodiment 36. The method of embodiment 33, wherein the tissue sample is a fresh tissue sample or a frozen tissue sample.
Embodiment 37. A kit, comprising:

(a) a plurality of stretching moieties;

(b) a plurality of primers;

(c) one or more enzymes selected from a polymerase, a reverse transcriptase, and a ligase;

(d) a substrate comprising a plurality of capture probes, wherein a capture probe of the plurality of capture probes comprises a spatial barcode and a capture domain; and

(e) instructions for performing the method of any one of the preceding embodiments.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining a presence or abundance of a nucleic acid analyte in a biological sample, the method comprising:
(a) providing the biological sample on a substrate comprising a plurality of capture probes, wherein a capture probe of the plurality of capture probes comprises a spatial barcode and a capture domain;
(b) releasing the nucleic acid analyte from the biological sample;
(c) affixing a stretching moiety to the nucleic acid analyte;
(d) hybridizing the nucleic acid analyte to the capture domain;
(e) applying a stretching force to the stretching moiety, thereby elongating the nucleic acid analyte hybridized to the capture domain; and
(f) generating an extended capture probe using the nucleic acid analyte as a template.

2. The method of claim 1, further comprising determining (i) all or a part of a sequence of the nucleic acid analyte or a complement thereof, and (ii) the spatial barcode or a complement thereof, and using the determined sequences of (i) and (ii) to determine the presence or abundance of the nucleic acid analyte in the biological sample.

3. The method of claim 1, wherein the stretching moiety is a magnetic bead, and wherein the stretching force is a magnetic force.

4. The method of claim 1, wherein the stretching force is a linear force orthogonal to a plane of an upper surface of the substrate, a rotational force around a rotational axis orthogonal to the plane of the upper surface of the substrate, or both.

5. The method of claim 1, wherein the affixing the stretching moiety to the nucleic acid analyte comprises affixing a first binding moiety to a second binding moiety,
wherein the stretching moiety comprises the first binding moiety, and
wherein the nucleic acid analyte comprises the second binding moiety associated with a 5′ end of the nucleic acid analyte or a 3′ end of the nucleic acid analyte.

6. The method of claim 5, wherein the first binding moiety and/or the second binding moiety comprises digoxigenin, anti-digoxigenin, biotin, avidin, or streptavidin.

7. The method of claim 1, wherein the stretching moiety further comprises a cleavable linker.

8. The method of claim 1, wherein the stretching force is applied using one of a magnetic field, an electric field, or a light field.

9. The method of claim 1, further comprising releasing the extended capture probe from the substrate.

10. The method of claim 1, wherein the releasing the nucleic acid analyte from the biological sample comprises treating the biological sample with a solution comprising pepsin or proteinase K.

11. The method of claim 1, wherein the capture domain comprises a poly(T) sequence.

12. The method of claim 1, wherein the nucleic acid analyte is RNA or DNA.

13. The method of claim 1, wherein the biological sample is a tissue sample.

14. The method of claim 1, wherein the tissue sample is a fixed tissue sample, a fresh tissue sample or a frozen tissue sample.

15. A method for determining a presence or abundance of a nucleic acid analyte in a biological sample, the method comprising:
(a) providing the biological sample on a substrate comprising a plurality of capture probes, wherein a capture probe of the plurality of capture probes comprises a spatial barcode and a capture domain;
(b) releasing the nucleic acid analyte from the biological sample;
(c) affixing a stretching moiety to the nucleic acid analyte;
(d) hybridizing the nucleic acid analyte to the capture domain;
(e) applying a stretching force to the stretching moiety, thereby elongating the nucleic acid analyte hybridized to the capture domain;
(f) hybridizing a padlock oligonucleotide to the nucleic acid analyte hybridized to the capture domain, wherein the padlock oligonucleotide comprises:
(i) a first sequence that is substantially complementary to a first portion of the nucleic acid analyte, or a complement thereof,
(ii) a backbone sequence, and
(iii) a second sequence that is substantially complementary to a second portion of the nucleic acid analyte, or a complement thereof;
(g) ligating the first sequence to the second sequence of the padlock oligonucleotide, thereby generating a circularized padlock oligonucleotide;
(h) amplifying the circularized padlock oligonucleotide, thereby creating an amplified circularized padlock oligonucleotide, and
(i) identifying the presence or abundance of the nucleic acid analyte in the biological sample.

16. The method of claim 15, wherein the identifying the presence or abundance of the nucleic acid analyte comprises determining (i) all or a part of a sequence of the nucleic acid analyte or a complement thereof, and (ii) the spatial barcode or a complement thereof, and using the determined sequences of (i) and (ii) to determine the presence or abundance of the nucleic acid analyte in the biological sample.

17. The method of claim 15, wherein the identifying the presence or abundance of the nucleic acid analyte comprises detecting a signal corresponding to the amplified circularized padlock oligonucleotide on the substrate.

18. The method of claim 17, further comprising quantitating the signal.

19. The method of claim 15, wherein the amplifying the circularized padlock oligonucleotide comprises rolling circle amplification.

20. The method of claim 15, wherein the first sequence of the padlock oligonucleotide and the second sequence of the padlock oligonucleotide are substantially complementary to adjacent sequences of the nucleic acid analyte.

21. The method of claim 15, wherein the first sequence of the padlock oligonucleotide and the second sequence of the padlock oligonucleotide are substantially complementary to sequences of the nucleic acid analyte that are not adjacent to one another, generating a gap between the first sequence and the second sequence upon hybridization of the first sequence and the second sequence to the nucleic acid analyte, wherein the gap is filled using a polymerase.

22. The method of claim 15, wherein the ligating step comprises enzymatic ligation or chemical ligation.

23. The method of claim 22, wherein the enzymatic ligation utilizes T4 DNA ligase.

24. The method of claim 15, wherein the stretching moiety is a magnetic bead, and wherein the stretching force is a magnetic force.

25. The method of claim 15, wherein the affixing the stretching moiety to the nucleic acid analyte comprises affixing a first binding moiety to a second binding moiety,
wherein the stretching moiety comprises the first binding moiety, and
wherein the nucleic acid analyte comprises the second binding moiety associated with a 5′ end of the nucleic acid analyte or a 3′ end of the nucleic acid analyte.

26. The method of claim 25, wherein the first binding moiety and/or the second binding moiety comprises digoxigenin, anti-digoxigenin, biotin, avidin, or streptavidin.

27. The method of claim 15, wherein the nucleic acid analyte is RNA or DNA.

28. The method of claim 15, wherein the biological sample is a tissue sample.

29. A method for determining a presence or abundance of a nucleic acid analyte in a biological sample, the method comprising:
(a) providing the biological sample on a first substrate;
(b) aligning the first substrate with a second substrate comprising an array, such that at least a portion of the biological sample is aligned with at least a portion of the array, wherein the array comprises a plurality of capture probes, wherein a capture probe of the plurality of capture probes comprises a spatial barcode and a capture domain;
(c) releasing the nucleic acid analyte from the biological sample, such that the nucleic acid analyte actively or passively migrates toward the capture probe, and binds the capture probe;
(d) affixing a stretching moiety to the nucleic acid analyte;
(e) hybridizing the nucleic acid analyte to the capture domain;
(f) applying a stretching force to the stretching moiety, thereby elongating the nucleic acid analyte hybridized to the capture domain; and
(g) extending the capture probe using the nucleic acid analyte as a template, thereby generating an extended capture probe.

30. The method of claim 29, wherein the nucleic acid analyte is RNA or DNA, and wherein the method further comprises determining (i) all or a part of a sequence of the nucleic acid analyte or a complement thereof, and (ii) the spatial barcode or a complement thereof, and using the determined sequences of (i) and (ii) to determine the presence or abundance of the nucleic acid analyte in the biological sample.
